(19) United States
(12) Patent Application Publication
(10) Pub. No.: US 2017/0066779 A1
(43) Pub. Date: Mar. 9, 2017

(54) SOLID FORMS OF A FLAVIVIRIDAE VIRUS INHIBITOR COMPOUND AND SALTS THEREOF

(71) Applicant: IDENIX Pharmaceuticals LLC, Cambridge, MA (US)

(72) Inventors: Adel M. Moussa, Boston, MA (US); Benjamin Alexander Mayes, Boston, MA (US); Alistair James Stewart, Somerville, MA (US)

(73) Assignee: IDENIX Pharmaceuticals LLC, Cambridge, MA (US)

(21) Appl. No.: 15/123,170

(22) PCT Filed: Mar. 4, 2015
(86) PCT No.: PCT/US2015/018571

§ 371 Date: Sep. 1, 2016

Related U.S. Application Data
(60) Provisional application No. 61/948,458, filed on Mar. 5, 2014.

(51) Int. Cl. C07D 495/04; A61K 31/4184; A61K 45/06

(52) U.S. Cl. CPC C07D 495/04; A61K 45/06; A61K 31/4184; C07B 2200/13

(57) ABSTRACT

Provided herein are crystalline and salt forms of methyl N-{(1R)-2-[(2S)-2-{5-[4-(6-{2-[(2S)-1-{(2S)-2-[(methoxycarbonyl)amino]-3-methylbutanoyl} pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl} carbamate, a Flaviviridae, including hepatitis C, virus inhibitor, pharmaceutical compositions comprising the compound, and processes of preparation thereof. Also provided are methods of its use for the treatment of a Flaviviridae, including HCV, infection in a subject in need thereof.

### FIELD

Provided herein are solid forms of a Flaviviridae virus inhibitor compound and salts thereof. Also provided herein are pharmaceutical compositions comprising the solid forms, and processes of their preparation. Further provided herein are methods of their use for the treatment of a flaviviridae, including hepatitis C, infection.

### BACKGROUND

The Flaviviridae family of viruses comprises at least three distinct genera: pestiviruses, which cause disease in cattle and pigs; Flaviviruses, which are the primary cause of diseases such as West Nile virus, dengue fever and yellow fever; and hepaciviruses, whose sole member is hepatitis C (HCV). The flavivirus genus includes more than 68 members separated into groups on the basis of serological relatedness (Calisher et al., J Gen. Virol, 1993, 70, 37-43). Clinical symptoms vary and include fever, encephalitis and hemorrhagic fever (Fields Virology, Editors: Fields, B. N., Knipe, D. M., and Howley, P. M., Lippincott-Raven Publishers, Philadelphia, Pa., 1996, Chapter 31, 931-959). Flaviviruses of global concern that are associated with human disease include the dengue hemorrhagic fever viruses (DHF), yellow fever virus, shock syndrome and Japanese encephalitis virus (Halstead, S. B., Rev. Infect. Dis., 1984, 6, 251-264; Halstead, S. B., Science, 239:476-481, 1988; Monath, T. P., New Eng. J Med, 1988, 319, 641-643).

Hepatitis C virus (HCV) is known to cause at least 80% of posttransfusion hepatitis and a substantial proportion of sporadic acute hepatitis (Kuo et al., Science 1989, 244, 362-364; Thomas, Curr. Top. Microbiol. Immunol. 2000, 25-41). Preliminary evidence also implicates HCV in many cases of “idiopathic” chronic hepatitis, “cryptogenic” cirrhosis, and probably hepatocellular carcinoma unrelated to other hepatitis viruses, such as hepatitis B virus (Di Besceglie et al., Scientific American, 1999, October, 80-85; Boyer et al., J. Hepatol. 2000, 32, 98-112).

HCV is an enveloped virus containing a positive-sense single-stranded RNA genome of approximately 9.4 kb (Kato et al., Proc. Natl. Acad. Sci. USA 1990, 87, 9524-9528; Kato, Acta Medica Okayama, 2001, 55, 133-159). The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as an internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

Recently sofosbuvir and simeprevir, in combination with interferon and/or ribavirin, were approved for treatment of adults with chronic HCV infection. Reardon, Nature, Oct. 30, 2013. These drugs boast cure rates of up to 80%, however the length of therapy and negative side effect are problematic. Previously, the most effective HCV therapy employed a combination of alpha-interferon and ribavirin, leading to sustained efficacy in about 40% of patients (Poynard et al., Lancet 1998, 352, 1426-1432). Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load (Manns et al., Lancet 2001, 358, 958-965; Fried et al., N. Engl. J. Med. 2002, 347, 975-982; Hadziyannis et al., Ann. Intern. Med. 2004, 140, 346-355). Currently, there are no known treatments for Yellow Fever, West Nile virus, Dengue fever, or Japanese encephalitis. Thus, there is a clear and unmet need to develop effective therapeutics for treatment of Flaviviridae, including HCV, infections.

### SUMMARY OF THE DISCLOSURE

Provided herein is a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate of Formula I:

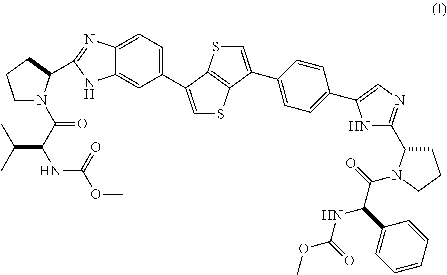

or an isotopic variant thereof; or a pharmaceutically acceptable salt or solvate thereof.

Also provided herein is a crystalline form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof.

Additionally provided herein is Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof.

Provided herein is a process for preparing a crystalline form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof comprising dissolving methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) in a solvent at a first temperature.

Provided herein is a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

Provided herein is a solid form of a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

Provided herein is a process for preparing a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof comprising contacting the compound with an acid.

Provided herein is a pharmaceutical composition comprising a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof, e.g., crystalline Form A; and a pharmaceutically acceptable excipient.

Provided herein is a pharmaceutical composition comprising a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof; and a pharmaceutically acceptable excipient.

Provided herein is a pharmaceutical composition comprising Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof; and a pharmaceutically acceptable excipient.

Provided herein is a pharmaceutical composition comprising a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof; and a pharmaceutically acceptable excipient.

Provided herein is a method for treating or preventing a Flaviviridae, including HCV, infection in a subject, comprising administering to a subject a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof, e.g., crystalline Form A.

Provided herein is a method for treating or preventing a Flaviviridae, including HCV, infection in a subject, comprising administering to a subject a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

Provided herein is a method for treating, preventing, or ameliorating one or more symptoms of a liver disease or disorder associated with a Flaviviridae, including HCV, infection in a subject, comprising administering to a subject a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof, e.g., crystalline Form A.

Provided herein is a method for treating, preventing, or ameliorating one or more symptoms of a liver disease or disorder associated with a Flaviviridae, including HCV, infection in a subject, comprising administering to a subject a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

Provided herein is a method for inhibiting replication of a Flaviviridae, including HCV, virus in a subject, comprising administering to a subject a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable salt or solvate thereof, e.g., crystalline Form A.

Provided herein is a method for inhibiting replication of a Flaviviridae, including HCV, virus in a subject, comprising administering to a subject a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

### BRIEF DESCRIPTION OF THE FIGURE

FIG. 1 depicts an exemplary X-ray powder (XRP) diffractogram of a sample of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) in crystalline Form A.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The term “isotopic variant” refers to a therapeutic agent that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent is in a stable form, that is, non-radioactive. In certain embodiments, an “isotopic variant” of a therapeutic agent contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a therapeutic agent is in an unstable form, that is, radioactive. In certain embodiments, an “isotopic variant” of a therapeutic agent contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a therapeutic agent, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 22nd ed.; Pharmaceutical Press: 2012; Handbook of Pharmaceutical Excipients, 7th ed.; Rowe et al., Eds.; The Pharmaceutical Press: 2012; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range. In certain embodiments, “about” or “approximately” with reference to X-ray powder diffraction two-theta peaks means within ±0.1° or ±0.2°.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer or an isotopic variant of a compound described herein.

The term “anti-solvent” refers to a liquid that is added to a solvent to reduce the solubility of a compound in that solvent, in some instances, resulting in precipitation of the compound.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The term “hepatitis C virus” or “HCV” refers to a viral species or a variant thereof, a pathogenic strain of which causes hepatitis C. Examples of HCV include, but are not limited to, HCV genotypes 1, 2, 3, 4, 5, 6, 7, 8, 9, 10, 11, and subtype 1a, 1b, 1c, 2a, 2b, 2c, 3a, 3b, 4a, 4b, 4c, 4d, 4e, 5a, 6a, 7a, 7b, 8a, 8b, 9a, 10a, and 11a. In certain embodiments, an HCV variant is an HCV species that contains a protein substantially homologous to a native HCV protein, i.e., a protein having one or more naturally or non-naturally occurring amino acid deletions, insertions or substitutions (e.g., derivatives, homologs, and fragments), as compared to the amino acid sequence of the native protein. The amino acid sequence of a protein of an HCV variant is at least about 80% identical, at least about 90% identical, or at least about 95% identical to a native HCV protein. In certain embodiments, the HCV variant contains an NS5A protein variant.

The term “NS5A” refers to nonstructural protein 5A or a variant thereof. NS5A variants include proteins substantially homologous to a native NS5A, i.e., proteins having one or more naturally or non-naturally occurring amino acid deletions, insertions or substitutions (e.g., NS5A derivatives, homologs, and fragments), as compared to the amino acid sequence of a native NS5A. The amino acid sequence of an NS5A variant is at least about 80% identical, at least about 90% identical, or at least about 95% identical to a native NS5A.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “crystalline form” of a compound can refer to any crystalline form of the compound as a free acid, the compound as a free base, as an acid addition salt of the compound, an base addition salt of the compound, a complex of the compound, a solvate (including hydrate) of the compound, or a co-crystal of the compound. The term “solid form” of a compound can refer to any crystalline form of the compound or any amorphous form of the compound as a free acid, the compound as a free base, as an acid addition salt of the compound, an base addition salt of the compound, a complex of the compound, or a solvate (including hydrate) of the compound, or a co-precipitation of the compound. In many instances, the terms “crystalline form” and “solid form” can refer to those that are pharmaceutically acceptable, including, for example, those of pharmaceutically acceptable addition salts, pharmaceutically acceptable complexes, pharmaceutically acceptable solvates, pharmaceutically acceptable co-crystals, and pharmaceutically acceptable co-precipitations.

### Solid Forms

In one embodiment, provided herein is a solid form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate or an isotopic variant thereof. For example, methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate can have the structure of Formula I:

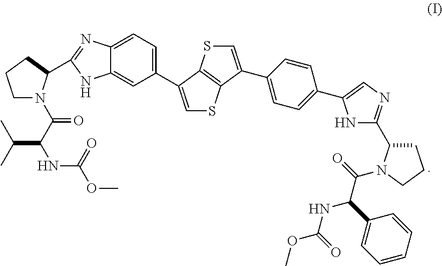

The compound of Formula I has been identified as a HCV inhibitor and can be prepared according to U.S. Pat. No. 8,362,068, the disclosure of which is incorporated herein by reference in its entirety.

In another embodiment, provided herein is a crystalline form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

In yet another embodiment, provided herein is a crystalline form of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof; wherein the crystalline form is Form A.

In various embodiments, crystalline Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) has an X-ray powder diffraction pattern. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 2.7°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 16.3°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 15.4°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 25.1°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 16.8°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 22.7°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 19.7°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 8.1°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 12.5°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 21.5°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 11.4°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 20.6°. In some embodiments, the X-ray-powder diffraction pattern of Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) includes an XRP diffraction peak at two-theta angles of approximately 23.5°.

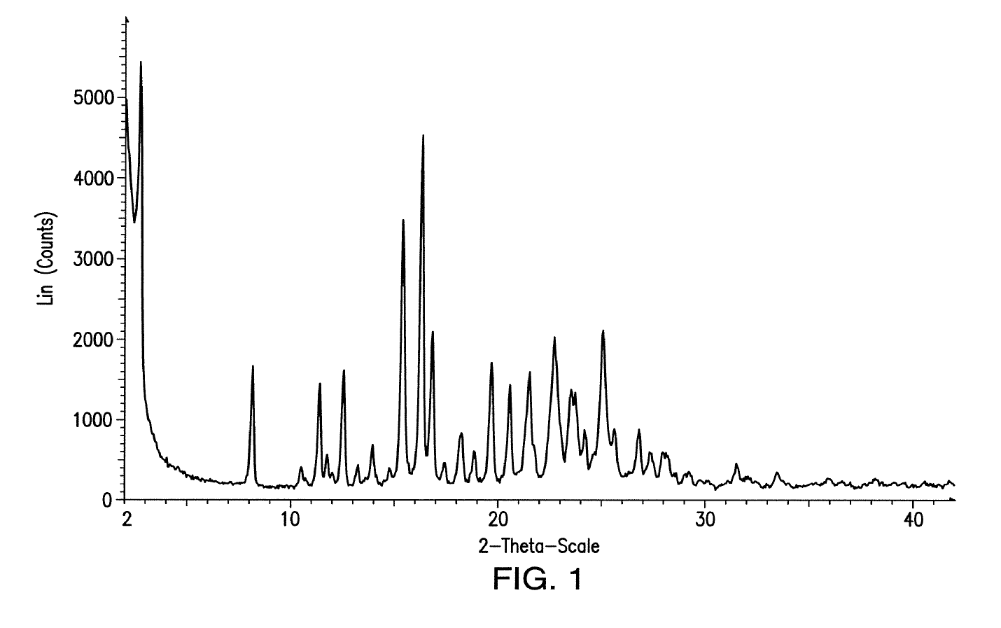

In certain embodiments, crystalline Form A has one or more characteristic XRP diffraction peaks at two-theta angles of approximately 2.7° and approximately 16.3°. In certain embodiments, crystalline Form A has one or more characteristic XRP diffraction peaks at two-theta angles of approximately 2.7°, approximately 15.4° and approximately 16.3°. In certain embodiments, crystalline Form A has one or more characteristic XRP diffraction peaks at two-theta angles of approximately 2.7°, approximately 15.4°, approximately 16.3°, and approximately 25.1°. In certain embodiments, crystalline Form A has one or more characteristic XRP diffraction peaks at two-theta angles of approximately 2.7°, approximately 15.4°, approximately 16.3°, approximately 16.8°, and approximately 25.1°. In certain embodiments, crystalline Form A has one or more characteristic XRP diffraction peaks at two-theta angles of approximately 2.7°, approximately 15.4°, approximately 16.3°, approximately 16.8°, approximately 22.7°, and approximately 25.1°. In certain embodiments, crystalline Form A has one or more characteristic XRP diffraction peaks at two-theta angles of approximately 2.7°, approximately 15.4°, approximately 16.3°, 16.8°, approximately 19.7°, approximately 22.7°, and approximately 25.1°. In certain embodiments, crystalline Form A has one or more characteristic XRP diffraction peaks at two-theta angles of approximately 2.7°, approximately 8.1°, approximately 15.4°, approximately 16.3°, approximately 16.8°, approximately 19.7°, approximately 22.7°, and approximately 25.1°. In certain embodiments, crystalline Form A has one or more characteristic XRP diffraction peaks at two-theta angles of approximately 2.7°, approximately 8.1°, approximately 12.5°, approximately 15.4°, approximately 16.3°, approximately 16.8°, approximately 19.7°, approximately 22.7°, and approximately 25.1°. In certain embodiments, crystalline Form A has one or more characteristic XRP diffraction peaks at two-theta angles of approximately 2.7°, approximately 8.1°, approximately 12.5°, approximately 15.4°, approximately 16.3°, approximately 16.8°, approximately 19.7°, approximately 21.5°, approximately 22.7°, and approximately 25.1°. In certain embodiments, crystalline Form A has one or more characteristic XRP diffraction peaks at two-theta angles of approximately 2.7°, approximately 8.1°, approximately 11.4°, approximately 12.5°, approximately 15.4°, approximately 16.3°, approximately 16.8°, approximately 19.7°, approximately 20.6°, approximately 21.5°, approximately 22.7°, approximately 23.5°, approximately 23.7°, and approximately 25.1°. In certain embodiments, crystalline Form A of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) has an X-ray powder diffraction pattern substantially as shown in FIG. 1.

In certain embodiments, crystalline Form A has an aqueous solubility of about 0.001, about 0.002, about 0.003, about 0.004, about 0.005, about 0.006, about 0.007, about 0.008, about 0.009, about 0.01, about 0.011, about 0.012, about 0.013, about 0.014, about 0.015, about 0.016, about 0.017, about 0.018, about 0.019, about 0.02, about 0.021, about 0.022, about 0.023, about 0.024, about 0.025, about 0.026, about 0.027, about 0.028, about 0.029, about 0.03, about 0.031, about 0.032, about 0.033, about 0.034, about 0.035, about 0.036, about 0.037, about 0.038, about 0.039, about 0.04, about 0.041, about 0.042, about 0.043, about 0.044, about 0.045, about 0.046, about 0.047, about 0.048, about 0.049, or about 0.05 mg/mL.

In certain embodiments, crystalline Form A may contain no less than about 90%, no less than about 91%, no less than about 92%, no less than about 93%, no less than about 94%, no less than about 95%, no less than about 96%, no less than about 97%, no less than about 98%, no less than about 98.2%, no less than about 98.5%, no less than about 98.7%, no less than 98.9%, no less than about 99%, or no less than about 99.5% by weight of the compound of Formula I.

In certain embodiments, crystalline Form A may contain no greater than about 0.1%, no greater than about 0.11%, no greater than about 0.12%, no greater than about 0.13%, no greater than about 0.14%, no greater than about 0.15%, no greater than about 0.16%, no greater than about 0.17%, no greater than about 0.18%, no greater than about 0.19%, no greater than about 0.2%, no greater than about 0.21%, no greater than about 0.22%, no greater than about 0.23%, no greater than about 0.24%, no greater than about 0.25%, no greater than about 0.26%, no greater than about 0.27%, no greater than about 0.28%, no greater than about 0.29%, no greater than about 0.3%, no greater than about 0.31%, no greater than about 0.32%, no greater than about 0.33%, no greater than about 0.34%, no greater than about 0.35%, no greater than about 0.36%, no greater than about 0.37%, no greater than about 0.38%, no greater than about 0.39%, no greater than about 0.4%, no greater than about 0.5%, no greater than about 0.6%, no greater than about 0.7%, no greater than about 0.8%, no greater than about 0.9%, no greater than about 1%, no greater than about 2%, no greater than about 3%, no greater than about 4%, or no greater than about 5% water by weight.

In various embodiments, crystalline Form A has an endotherm a differential scanning calorimetric thermogram. In some embodiments, crystalline Form A has an onset temperature above about 150° C. in a differential scanning calorimetric thermogram. In some embodiments, crystalline Form A has an onset temperature above about 180° C. in a differential scanning calorimetric thermogram. In some embodiments, crystalline Form A has an onset temperature above about 200° C. in a differential scanning calorimetric thermogram. In some embodiments, crystalline Form A has an onset temperature above about 220° C. in a differential scanning calorimetric thermogram. In some embodiments, crystalline Form A has an onset temperature above about 240° C. in a differential scanning calorimetric thermogram. In some embodiments, crystalline Form A has an onset temperature above about 242° C. in a differential scanning calorimetric thermogram. In some embodiments, crystalline Form A has an onset temperature above about 244° C. in a differential scanning calorimetric thermogram. In some embodiments, crystalline Form A has an onset temperature above about 245° C. in a differential scanning calorimetric thermogram. In certain embodiments, crystalline Form A has an onset temperature between about 200° C. to about 280° C. in a differential scanning calorimetric thermogram. In certain embodiments, crystalline Form A has an onset temperature between about 220° C. to about 260° C. in a differential scanning calorimetric thermogram. In certain embodiments, crystalline Form A has an onset temperature between about 240° C. to about 250° C. in a differential scanning calorimetric thermogram. In certain embodiments, crystalline Form A has an onset temperature between about 245° C. to about 250° C. in a differential scanning calorimetric thermogram. In certain embodiments, crystalline Form A has an onset temperature of about 247° C. in a differential scanning calorimetric thermogram.

In some embodiments, crystalline Form A has a peak temperature above about 150° C. in a differential scanning calorimetric thermogram. In some embodiments, crystalline Form A has a peak temperature above about 180° C. in a differential scanning calorimetric thermogram. In some embodiments, crystalline Form A has a peak temperature above about 200° C. in a differential scanning calorimetric thermogram. In some embodiments, crystalline Form A has a peak temperature above about 220° C. in a differential scanning calorimetric thermogram. In some embodiments, crystalline Form A has a peak temperature above about 240° C. in a differential scanning calorimetric thermogram. In some embodiments, crystalline Form A has a peak temperature above about 245° C. in a differential scanning calorimetric thermogram. In some embodiments, crystalline Form A has a peak temperature above about 250° C. in a differential scanning calorimetric thermogram. In some embodiments, crystalline Form A has a peak temperature between about 150° C. and about 300° C. in a differential scanning calorimetric thermogram. In some embodiments, crystalline Form A has a peak temperature between about 200° C. and about 290° C. in a differential scanning calorimetric thermogram. In some embodiments, crystalline Form A has a peak temperature between about 220° C. and about 280° C. in a differential scanning calorimetric thermogram. In some embodiments, crystalline Form A has a peak temperature between about 240° C. and about 275° C. in a differential scanning calorimetric thermogram. In some embodiments, crystalline Form A has a peak temperature between about 245° C. and about 270° C. in a differential scanning calorimetric thermogram. In some embodiments, crystalline Form A has a peak temperature between about 245° C. and about 265° C. in a differential scanning calorimetric thermogram. In certain embodiments, crystalline Form A has an endotherm with a peak temperature of about 253° C. in a differential scanning calorimetric thermogram. In certain embodiments, crystalline Form A has an endotherm with a peak temperature of about 253° C. and an onset temperature of about 247° C. in a differential scanning calorimetric thermogram.

In certain embodiments, crystalline Form A has a weight loss of no greater than about 3%, no greater than about 2.9%, no greater than about 2.8%, no greater than about 2.7%, no greater than about 2.6%, no greater than about 2.5%, no greater than about 2.4%, no greater than about 2.3%, no greater than about 2.2%, no greater than about 2.1%, no greater than about 2.0%, no greater than about 1.9%, no greater than about 1.8%, no greater than about 1.7%, no greater than about 1.6%, no greater than about 1.5%, no greater than about 1.4%, no greater than about 1.3%, no greater than about 1.2%, no greater than about 1.1%, no greater than about 1.0%, no greater than about 0.9%, no greater than about 0.8%, no greater than about 0.7%, no greater than about 0.6%, no greater than about 0.5%, no greater than about 0.4%, no greater than about 0.3%, no greater than about 0.2%, or no greater than about 0.1% between about 25 and about 250° C. in a thermogravimetric thermogram.

In certain embodiments, crystalline Form A has a residual solvent content of no greater than about 10%, no greater than about 8%, no greater than about 7%, no greater than about 5%, no greater than about 2%, no greater than about 1%, no greater than about 0.9%, no greater than about 0.8%, no greater than about 0.7%, no greater than about 0.6%, no greater than about 0.5%, no greater than about 0.4%, no greater than about 0.3%, no greater than about 0.2%, no greater than about 0.1%, no greater than about 0.05%, or no greater than about 0.01% by weight. In certain embodiments, the solvent is acetone, ethyl acetate, dioxane, transcutol, tetralin, diisopropyl ether, diethyl ether, toluene, MTBE, anisole, isopropanol, isopropyl acetate, THF, dichloromethane, methyl-ethyl ketone, methanol, ethanol, acetonitrile, nitromethane, water, or a mixture thereof.

In one embodiment, provided herein is a solid form of a pharmaceutical salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I).

### Method of Preparation

In one embodiment, a process for the preparation of crystalline Form A comprises dissolving the compound of Formula I in a solvent. For example, the dissolving of the compound of Formula I in a solvent is at a first temperature.

In one embodiment, the process for the preparation of crystalline Form A comprises dissolving the compound of Formula I in a solvent, wherein the ratio of the weight in grams of the compound of Formula I to the volume of solvent in milliliters is about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1.

In one embodiment, the process for the preparation of crystalline Form A comprises dissolving the compound of Formula I in a first solvent, wherein the first solvent is selected from acetone, ethyl acetate, dioxane, transcutol, tetralin, diethyl ether, diisopropyl ether, toluene, MTBE, anisole, isopropanol, isopropyl acetate, THF, dichloromethane, methyl-ethyl ketone, methanol, ethanol, acetonitrile, nitromethane, water, or a mixture thereof.

In one embodiment, the process for the preparation of crystalline Form A comprises dissolving the compound of Formula I in a first solvent, wherein the first solvent is selected from acetone, ethyl acetae, dioxane, transcutol, tetralin, diisopropyl ether, diethyl ether, MTBE, dichloromethane, ethanol, methanol, toluene, water, or a mixture thereof.

In one embodiment, the process for the preparation of crystalline Form A comprises dissolving the compound of Formula I in a first solvent, wherein the first solvent is acetone, methanol, dichloromethane, toluene, or a mixture thereof.

In one embodiment, the process for the preparation of crystalline Form A comprises dissolving the compound of Formula I in a first temperature, wherein the first temperature is about 0, about 5, about 10, about 15, about 20, about 25, about 30, about 35, about 40, about 45, about 50, about 55, about 60, about 65, about 70, about 75, about 80, about 85, about 90, about 95, or about 100° C.

In one embodiment, the process for the preparation of crystalline Form A comprises dissolving the compound of Formula I in a first temperature, wherein the first temperature is about 50° C. or about 60° C.

In one embodiment, the process for the preparation of crystalline Form A comprises adding a second solvent, wherein the second solvent is selected from hexane, heptanes, octane, acetone, ethyl acetate, dioxane, transcutol, tetralin, diethyl ether, diisopropyl ether, toluene, MTBE, anisole, isopropanol, isopropyl acetate, THF, dichloromethane, methyl-ethyl ketone, methanol, ethanol, acetonitrile, nitromethane, water, or a mixture thereof.

In one embodiment, the process for the preparation of crystalline Form A comprises adding a second solvent, wherein the second solvent is selected from hexane, heptanes, octane, acetone, ethyl acetate, diisopropyl ether, toluene, MTBE, isopropanol, isopropyl acetate, THF, dichloromethane, methyl-ethyl ketone, methanol, ethanol, acetonitrile, nitromethane, water, or a mixture thereof. In some embodiments, the second solvent is acetone, ethyl acetate, diisopropyl ether, MTBE, isopropanol, isopropyl acetate, THF, dichloromethane, methyl-ethyl ketone, methanol, ethanol, acetonitrile, nitromethane, water, or a mixture thereof. In some embodiments, the second solvent is hexane, heptanes, octane, toluene, or a mixture thereof.

In one embodiment, the process of the preparation of crystalline Form A comprises removing at least part of the first solvent or the second solvent. For example, the removal of the first solvent or the second solvent can be achieved by distillation (e.g., at the embient temperature or a reduced pressure), decantation, or other techniques known in the art. In one embodiments, the removal of at least part of a solvent and/or the additional of another solvent is achieved separately, sequentially, or simultaneously. For example, the removal of at least part of a solvent and/or the additional of another solvent can be achieved sequentially or simultaneously, including by solvent swap.

In one embodiment, the process for the preparation of crystalline Form A comprises changing the solution to a second temperature.

In one embodiment, the process for the preparation of crystalline Form A comprises changing the solution to a second temperature, wherein the second temperature is about 0, about 5, about 10, about 15, about 20, about 25, about 30, about 35, about 40, about 45, about 50, about 55, about 60, about 65, about 70, about 75, about 80, about 85, about 90, or about 95° C.

In one embodiment, the process for the preparation of crystalline Form A comprises changing the solution to a second temperature, wherein the second temperature is about 20° C.

In one embodiment, the process for the preparation of crystalline Form A comprises adding one or more additives. For example, the one or more additives can be absorbents or filter aids. In some embodiments, the one or more additives can be silicas, aluminas, activated charcoals, florisils, molecular sieves, diatomaceous earths, exchange resins, or mixtures thereof. In one embodiment, the process for the preparation of crystalline Form A comprises filtering the mixture.

In one embodiment, the process for the preparation of crystalline Form A comprises drying the crystals at a drying temperature, for example, at approximately the ambient pressure or under a reduced pressure.

In one embodiment, the process for the preparation of crystalline Form A comprises drying the crystals at a drying temperature. In some embodiments, the drying temperature is no greater than about 100° C., no greater than about 95° C., no greater than about 90° C., no greater than about 85° C., no greater than about 80° C., or no greater than about 75° C. In particular embodiments, the drying temperature is no greater than about 75° C. Thus, in one particular embodiment, the process for the preparation of crystalline Form A comprises drying the crystals at a drying temperature, no greater than about 75° C. For example, the drying temperature can be from about 70° C. to about 80° C.

In one embodiment, the process for the preparation of crystalline Form A comprises milling the crystals.

### Pharmaceutically Acceptable Salts

In one embodiment, provided herein is a pharmaceutically acceptable salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

In certain embodiments, the pharmaceutically acceptable salt provided herein has a purity of at least about 50%, at least about 70%, at least about 80%, at least about 90%, at least about 95%, at least about 96%, at least about 97%, at least about 98%, at least about 98.5%, at least about 99%, at least about 99.2%, at least about 99.4%, at least about 99.5%, at least 99.6%, at least 99.7%, at least 99.8%, or at least 99.9% by weight.

In another embodiment, provided herein is a pharmaceutically acceptable acid addition salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof.

In one embodiment, the acid used to prepare a pharmaceutically acceptable acid addition salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof; or a pharmaceutically acceptable solvate thereof is selected from HI, HBr, HCl, HF, H₂SO₄, p-toluene sulfonic acid, methanesulfonic acid, benzenesulfonic acid, oxalic acid, L-aspartic acid, maleic acid, ketoglutaric acid, malonic acid, thiocyanic acid, L-tartaric acid, fumaric acid, citric acid, L-malic acid, D-gluconic acid, L-lactic acid, L-ascorbic acid, benzoic acid, nicotinic acid, glycolic acid, camphorsulfonic acid, sucrose, or nicotinamide. For example, the acid can be selected from HBr, HCl, H₂SO₄, toluene sulfonic acid, methanesulfonic acid, or benzenesulfonic acid.

In another embodiment, the molar ratio of the acid in the acid addition salt versus methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) or an isotopic variant thereof is about 0.5, about 1, about 1.5, about 2, about 2.5, or about 3. For example, the molar ratio can be from about 0.5 to about 3, from about 0.5 to about 2.5, from about 0.5 to about 2, from about 0.5 to about 1.5, from about 1 to about 3, from about 1 to about 2.5, from about 1 to about 2, from about 1 to about 1.5.

In one embodiment, the acid addition salt is a hydrochloride salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I). In one embodiment, the hydrochloride salt is an amorphous solid. In another embodiment, the molar ratio is about 0.5, about 1, about 1.5, about 2, about 2.5, or about 3.

In yet another embodiment, the acid addition salt is a sulfuric acid salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I).

In yet another embodiment, the acid addition salt is a toluenesulfonic acid salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I).

In yet another embodiment, the acid addition salt is a methanesulfonic acid salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I).

In still another embodiment, the acid addition salt is a benzenesulfonic acid salt of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I).

In one embodiment, provided herein is a pharmaceutically-acceptable salt of a compound of the compound of Formula I or a solvate thereof.

In one embodiment, the pharmaceutically-acceptable salt is an acid addition salt. In one embodiment, the acid addition salt is crystalline or amorphous.

In one embodiment, the acid of the acid addition salt is selected from HI, HBr, HCl, HF, H₂SO₄, p-toluene sulfonic acid, methanesulfonic acid, benzenesulfonic acid, oxalic acid, L-aspartic acid, maleic acid, ketoglutaric acid, malonic acid, thiocyanic acid, L-tartaric acid, fumaric acid, citric acid, L-malic acid, D-gluconic acid, L-lactic acid, L-ascorbic acid, benzoic acid, nicotinic acid, glycolic acid, camphorsulfonic acid, sucrose, or nicotinamide.

In one embodiment, the acid addition salt comprising of about 0.5, about 1, about 1.5, about 2, about 2.5, or about 3 molar equivalents of acid and about one molar equivalent of the compound of Formula I.

In certain embodiments, the acid addition salt has an aqueous solubility of about 0.000, about 0.01, about 0.02, about 0.03, about 0.04, about 0.05, about 0.06, about 0.07, about 0.08, about 0.09, about 0.1, about 1, about 1.2, about 1.3, about 1.4, about 1.5, about 1.6, about 1.7, about 1.8, about 1.9, or about 2 mg/mL.

In certain embodiments, the acid addition salt may contain no greater than about 0.5%, no greater than about 0.6%, no greater than about 0.7%, no greater than about 0.8%, no greater than about 0.9%, no greater than about 1%, no greater than about 1.3%, no greater than about 1.5%, no greater than about 1.7%, no greater than about 1.9%, no greater than about 2%, no greater than about 2.3%, no greater than about 2.5%, no greater than about 2.7%, no greater than about 2.9%, no greater than about 3%, no greater than about 4%, or no greater than about 5% water by weight.

In certain embodiments, the acid addition salt has a weight loss of no greater than about 3%, no greater than about 3.5%, no greater than about 4%, no greater than about 4.5%, no greater than about 4.6%, no greater than about 5%, no greater than about 5.2%, or no greater than about 5.5% in a thermogravimetric thermogram.

In certain embodiments, the acid addition salt may contain no less than about 95%, no less than about 97%, no less than about 98%, no less than about 98.2%, no less than about 98.5%, no less than about 98.7%, no less than 98.9%, no less than about 99%, or no less than about 99.5% by weight of the compound of Formula I.

In one embodiment, provided herein is a process for the preparation of a pharmaceutically-acceptable salt, comprising reacting a compound of Formula I with an acid in a solvent at an elevated temperature.

In one embodiment, the process for the preparation of a pharmaceutically-acceptable salt comprises reacting a compound of Formula I with an acid in a solvent at an elevated temperature, wherein the elevated temperature is 50° C.

In one embodiment, the process for the preparation of a pharmaceutically-acceptable salt comprises precipitating the salt at a second temperature.

In one embodiment, the process for the preparation of a pharmaceutically-acceptable salt comprises precipitating the salt at a second temperature, wherein the second temperature is 0° C.

In one embodiment, the solvent in the process for the preparation of a pharmaceutically-acceptable salt is selected from acetone, ethyl acetate, dioxane, transcutol, tetralin, diisopropyl ether, diethyl ether, toluene, MTBE, anisole, isopropanol, isopropyl acetate, THF, dichloromethane, methyl-ethyl ketone, methanol, ethanol, acetonitrile, nitromethane, water, or a mixture thereof.

In one embodiment, the process for the preparation of a pharmaceutically-acceptable salt comprises reacting a compound of Formula I with an acid, wherein the acid is selected from HI, HBr, HCl, HF, H₂SO₄, p-toluene sulfonic acid, methanesulfonic acid, benzenesulfonic acid, oxalic acid, L-aspartic acid, maleic acid, ketoglutaric acid, malonic acid, thiocyanic acid, L-tartaric acid, fumaric acid, citric acid, L-malic acid, D-gluconic acid, L-lactic acid, L-ascorbic acid, benzoic acid, nicotinic acid, glycolic acid, camphorsulfonic acid, sucrose, or nicotinamide.

### Pharmaceutical Compositions

In one embodiment provided herein are pharmaceutical compositions which comprise solid methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate (Formula I) in a crystalline form or a pharmaceutically acceptable salt, in combination with one or more pharmaceutically acceptable carriers or excipients. The choice of excipient, to a large extent, depends on factors, such as the particular mode of administration, the effect of the excipient on the solubility and stability of the active ingredient, and the nature of the dosage form.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by a pharmaceutical composition comprising a crystalline or salt form of the compound of Formula I, or a pharmaceutically acceptable solvate or prodrug thereof, provided herein, in combination or alternation with a second, and perhaps third, antiviral compound that induces a different mutation from that caused by the principal drug. Alternatively, the pharmacokinetics, biodistribution, or other parameters of the drug can be altered by such combination or alternation therapy. In general, combination therapy is typically preferred over alternation therapy because it induces multiple simultaneous stresses on the virus.

In one embodiment, a pharmaceutical composition comprises a crystalline or salt form of the compound of Formula I and one or more pharmaceutically acceptable carriers. In one embodiment, a pharmaceutical composition further comprises a second antiviral agent. In one embodiment, the second antiviral agent is selected from the group consisting of an interferon, ribavirin, amantadine, an interleukin, an NS3 protease inhibitor, an NS5A inhibitor, an NS5B inhibitor, a cyclophilin inhibitor, a cysteine protease inhibitor, a phenanthrenequinone, a thiazolidine, a benzanilide, a helicase inhibitor, a polymerase inhibitor, a nucleotide analogue, a nucleoside analogue, a gliotoxin, a cerulenin, an antisense phosphorothioate oligodeoxynucleotide, an inhibitor of IRES-dependent translation, or a ribozyme. In one embodiment, the second antiviral agent is an interferon. In another embodiment, the interferon is selected from the group consisting of pegylated interferon alpha 2a, interferon alfacon-1, natural interferon, ALBUFERON®, interferon beta-1a, omega interferon, interferon alpha, interferon gamma, interferon tau, interferon delta, or interferon gamma-1b.

In certain embodiments, a pharmaceutical composition comprising a crystalline or salt form of the compound of Formula I, or a pharmaceutically acceptable solvate or prodrug thereof, is combined with a cyclophilin inhibitor, including, but not limited to, alisporivir (Novartis), cyclosporin A, sanglifehrins and sanglifehrin analogs, CsD, NIM-811, and SCY-635.

In certain embodiments, a pharmaceutical composition comprising a crystalline or salt form of the compound of Formula I, or a pharmaceutically acceptable solvate or prodrug thereof, is combined with an NS5A inhibitor, including, but not limited to, ABT-267, BMS-790052, GS-5885, GS-5816, PPI-461, and PPI-668.

In certain embodiments, a pharmaceutical composition comprising a crystalline or salt form of the compound of Formula I, or a pharmaceutically acceptable solvate or prodrug thereof, is combined with an NS5B inhibitor, including, but not limited to, ABT-072, ABT-333, ANA598, BI 207127, GS-9669, GS-9190, GSK-625433, HCV-796, IDX184, IDX375, IDX19368, JTK-109, MK-0608, MK-3281, NM283, PF-868554, PSI-879, PSI-938, PSI-6130, PSI-7851, PSI-7977, R1626, R7128, VCH-222, VCH-759, and VCH-916. In one embodiment, the NS5B inhibitor is PSI-7977 (sofosbuvir). In another embodiment, the NS5B inhibitor is one or more NS5B inhibitors described in U.S. Patent Publication No. 2013-0315868, hereby incorporated by reference herein, it its entirety.

In certain embodiments, a pharmaceutical composition comprising a crystalline or salt form of the compound of Formula I, or a pharmaceutically acceptable solvate or prodrug thereof, is combined with an HCV protease inhibitor, including, but not limited to, ACH-0141625, faldaprevir (BI 201335); asunaprevir (BMS-650032); TMC 435 or TMC 435350 (Medivir/Tibotec); ITMN 191/R7227 (InterMune); MK 7009 (Merck); SCH 5034/SCH 503034/Boceprevir and SCH 900518/narlaprevir (Schering); VX950/telaprevir (Vertex); substrate-based NS3 protease inhibitors as disclosed in (DE 19914474, WO 98/17679, WO 98/22496, WO 99/07734, and Attwood et al., Antiviral Chemistry and Chemotherapy 1999, 10, 259-273); non-substrate-based NS3 protease inhibitors, including 2,4,6-trihydroxy-3-nitro-benzamide derivatives (Sudo et al., Biochem. Biophys. Res. Commun. 1997, 238, 643-647), a phenanthrenequinone (Chu et al., Tetrahedron Letters 1996, 37, 7229-7232), RD3-4082, RD3-4078, SCH 68631, and SCH 351633 (Chu et al., Bioorganic and Medicinal Chemistry Letters 1999, 9, 1949-1952); and Eglin C, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinás-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,608,027; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; and 7,491,794; U.S. Pat. App. Pub. Nos.: 2002/0016294, 2002/0016442; 2002/0032175; 2002/0037998; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781, 2008/0152622, 2009/0035271, 2009/0035272, 2009/0047244, 2009/0111969, 2009/0111982, 2009/0123425, 2009/0130059, 2009/0148407, 2009/0156800, 2009/0169510, 2009/0175822, 2009/0180981, and 2009/0202480; U.S. patent application Ser. No. 12/365,127; and International Pat. App. Pub. Nos.: WO 98/17679; WO 98/22496; WO 99/07734; WO 00/09543; WO 00/59929; WO 02/08187; WO 02/08251; WO 02/08256; WO 02/08198; WO 02/48116; WO 02/48157; WO 02/48172; WO 02/60926; WO 03/53349; WO 03/64416; WO 03/64455; WO 03/64456; WO 03/66103; WO 03/99274; WO 03/99316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; WO 2007/015824, WO 2007/056120, WO 2008/019289, WO 2008/021960, WO 2008/022006, WO 2008/086161, WO 2009/053828, WO 2009/058856, WO 2009/073713, WO 2009/073780, WO 2009/080542, WO 2009/082701, WO 2009/082697, and WO 2009/085978; the disclosure of each of which is incorporated herein by reference in its entirety.

Other protease inhibitors include thiazolidine derivatives, such as RD-1-6250, RD4 6205, and RD4 6193, which show relevant inhibition in a reverse-phase HPLC assay with an NS3/4A fusion protein and NS5A/5B substrate (Sudo et al., Antiviral Research 1996, 32, 9-18); and thiazolidines and benzanilides identified in (Kakiuchi et al., FEBS Lett. 1998, 421, 217-220; and Takeshita et al., Analytical Biochemistry 1997, 247, 242-246).

Suitable helicase inhibitors include, but are not limited to, those disclosed in U.S. Pat. No. 5,633,358; and International Pat. App. Pub. No. WO 97/36554.

Suitable nucleotide polymerase inhibitors include, but are not limited to, 2′-methyl ribofuranosyl nucleotides. See, e.g., WO 01/90121, WO 01/92282, WO 2004/002999, WO 2005/003147, U.S. Pat. Nos. 6,914,054; 7,608,597; 7,608,600; 7,824,851; 7,157,441; 7,635,689; 7,429,572; 7,754,699; 7,964,580; 7,105,499; 6,777,395; 8,481,712. In one embodiment, a nucleotide polymerase inhibitor is gliotoxin (Ferrari et al., Journal of Virology 1999, 73, 1649-1654), cerulenin (Lohmann et al., Virology 1998, 249, 108-118), ABT-072, ABT-333, AG-02154, ANA598, ANA773, GS-9190, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, R7128, RG7128, VCH-759, VCH-916 or VX-222 (VCH-222).

Suitable interfering RNA (iRNA) based antivirals include, but are not limited to, short interfering RNA (siRNA) based antivirals, such as Sirna-034 and those described in International Pat. App. Pub. Nos. WO/03/070750 and WO 2005/012525, and U.S. Pat. App. Pub. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; and Galderisi et al., Journal of Cellular Physiology 1999, 181, 251-257).

Suitable inhibitors of IRES-dependent translation include, but are not limited to, those described in Japanese Pat. App. Pub. Nos.: JP 08268890 and JP 10101591.

Suitable ribozymes include those disclosed in, for example, U.S. Pat. Nos. 6,043,077; 5,869,253; and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. App. Pub. Nos. 2004/0121980; 2005/0009737; 2005/0038240; and 2006/0040890; and International Pat. App. Pub. Nos: WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second antiviral agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, a pharmaceutical composition comprising a crystalline or salt form of the compound of Formula I, or a pharmaceutically acceptable solvate or prodrug thereof, is administered in combination or alternation with an anti-hepatitis C virus interferon, including, but not limited to, INTRON® A (interferon alfa-2b), PEGASYS® (Peginterferon alfa-2a) ROFERON® A (recombinant interferon alfa-2a), INFERGEN® (interferon alfacon-1), or PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, a pharmaceutical composition comprising a crystalline or salt form of the compound of Formula I, or a pharmaceutically acceptable solvate or prodrug thereof, is administered in combination or alternation with an anti-hepatitis C virus polymerase inhibitor, such as ribavirin, viramidine, NM 283 (valopicitabine), ABT-072, ABT-267, ABT-333, AG-02154, ANA598, ANA773, EDP-239, deleobuvir (BI 207127), GS-5885, GS-5816, GS-9190, HCV-796, IDX184, IDX375, JTK-109, MK-0608, MK-3281, NM283, PF-868554, PPI-668, PSI-879, PSI-938, PSI-6130, PSI-7851, sofosbuvir (PSI-7977), R1626, R7128, RG7128, VCH-759, VCH-916, VX-222 (VCH-222), or those as disclosed in U.S. Pat. App. Pub. Nos. 2009/0081158 and 2009/0238790, the disclosure of each of which is incorporated herein by reference in its entirety.

In certain embodiments, a pharmaceutical composition comprising a crystalline or salt form of the compound of Formula I, or a pharmaceutically acceptable solvate or prodrug thereof, is administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), or PEG-INTRON® (pegylated interferon alfa-2b).

In certain embodiments, a pharmaceutical composition comprising a crystalline or salt form of the compound of Formula I, or a pharmaceutically acceptable solvate or prodrug thereof, is administered in combination or alternation with an anti-hepatitis C virus protease inhibitor, such as ABT-450, ITMN-191, SCH 503034, VX950 (telaprevir), or TMC 435.

In certain embodiments, a pharmaceutical composition comprising a crystalline or salt form of the compound of Formula I, or a pharmaceutically acceptable solvate or prodrug thereof, is administered in combination or alternation with an anti-hepatitis C virus vaccine, including, but not limited to, TG4040, PEVIPRO™, CGI-5005, HCV/MF59, GV1001, IC41, or INNO0101 (E1).

In certain embodiments, a pharmaceutical composition comprising a crystalline or salt form of the compound of Formula I, or a pharmaceutically acceptable solvate or prodrug thereof, is administered in combination or alternation with an anti-hepatitis C virus monoclonal antibody, such as AB68 and XTL-6865 (formerly HepX-C); or an anti-hepatitis C virus polyclonal antibody, such as CIVACIR®.

In certain embodiments, a pharmaceutical composition comprising a crystalline or salt form of the compound of Formula I, or a pharmaceutically acceptable solvate or prodrug thereof, is administered in combination or alternation with an anti-hepatitis C virus immunomodulator, such as ZADAXIN® (thymalfasin), NOV-205, or oglufanide.

In certain embodiments, a pharmaceutical composition comprising a crystalline or salt form of the compound of Formula I, or a pharmaceutically acceptable solvate or prodrug thereof, is administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide), or PYN17.

The compounds solid form (including Form A), salt, or compositions provided herein can also be administered in combination with other classes of compounds, including, but not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporine; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (2S) Factor VIIa Inhibitors and Factor Xa Inhibitors; (26) farnesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

The solid form (including Form A), salt, or compositions compound provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In one embodiment, a pharmaceutical composition comprising a crystalline or salt form of the compound of Formula I formulated for single dose administration.

In one embodiment, a pharmaceutical composition comprising a crystalline or salt form of the compound of Formula I is formulated as an oral, parenteral, or intravenous dosage form. In one embodiment, the oral dosage form is a tablet or capsule.

In one embodiment, a pharmaceutical composition comprising a crystalline or salt form of the compound of Formula I administered in a dose of about 0.5 milligrams to about 1,000 milligrams daily.

In one embodiment, a method for treating or preventing an HCV infection in a subject, which comprises administering to a subject a crystalline or salt form of the compound of Formula I. In one embodiment, the subject is a human.

In one embodiment, a method of treating, preventing, or ameliorating one or more symptoms of a liver disease or disorder associated with an HCV infection in a subject, comprising administering to a subject a crystalline or salt form of the compound of Formula I. In another embodiment, the method comprises administering to the subject a second antiviral agent, in combination or alternation. In one embodiment, the second antiviral agent is selected from an interferon, ribavirin, amantadine, an interleukin, a NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, or a ribozyme. In one embodiment, the second antiviral agent is an interferon. In one embodiment, the interferon is selected from pegylated interferon alpha 2a, interferon alfacon-1, natural interferon, albuferon, interferon beta-1a, omega interferon, interferon alpha, interferon gamma, interferon tau, interferon delta, or interferon gamma-1b.

Provided herein are pharmaceutical compositions comprising a crystalline or salt form of the compound of Formula I, as an active ingredient, or an isotopic variant thereof; or a pharmaceutically acceptable solvate or prodrug thereof; or a pharmaceutically acceptable salt, solvate, hydrate, or prodrug; in combination with a pharmaceutically acceptable vehicle, carrier, diluent, or excipient, or a mixture thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose, or other mono- or di-saccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

The solid form (including Form A), salt, or compositions provided herein may be administered alone, or in combination with one or more other compounds provided herein. The pharmaceutical compositions that comprise a crystalline or salt form of the compound of Formula I, or an isotopic variant thereof; or a pharmaceutically acceptable solvate or prodrug thereof, can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

In one embodiment, the pharmaceutical compositions are provided in a dosage form for oral administration, which comprise a crystalline or salt form of the compound of Formula I, or an isotopic variant thereof; or a pharmaceutically acceptable solvate or prodrug thereof; and one or more pharmaceutically acceptable excipients or carriers.

In another embodiment, the pharmaceutical compositions are provided in a dosage form for parenteral administration, which comprise a crystalline or salt form of the compound of Formula I, or an isotopic variant thereof; or a pharmaceutically acceptable solvate or prodrug thereof; and one or more pharmaceutically acceptable excipients or carriers.

In yet another embodiment, the pharmaceutical compositions are provided in a dosage form for topical administration, which comprise a crystalline or salt form of the compound of Formula I, or an isotopic variant thereof; or a pharmaceutically acceptable solvate or prodrug thereof; and one or more pharmaceutically acceptable excipients or carriers.

The pharmaceutical compositions provided herein can be provided in a unit-dosage form or multiple-dosage form. A unit-dosage form, as used herein, refers to a physically discrete unit suitable for administration to a human and animal subject, and packaged individually as known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, or syrups. In addition to the active ingredient(s), the pharmaceutical compositions can contain one or more pharmaceutically acceptable carriers or excipients, each independently selected from, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, or sources of carbon dioxide.

The desired route of administration for the compound of Formula I is oral. The absolute oral bioavailability in rats was determined to be approximately 4-8% when dosed as a solution in 0.5% TPGS/37.5% PEG200/60% PBS. The doses were 10 mg/kg PO and 2 mg/kg IV.

The compound of Formula I is classified as BCS Class IV based on poor aqueous solubility (<0.5 mg/mL) and low permeability in the 3-day Caco-2 cell model. The compound of Formula I was not a P-gp substrate, as deduced from the BA/AB transport ratio of <1 and absence of effect from addition of P-gp inhibitor cyclosporine A.

Furthermore, the half-life in human hepatocytes was in the mid/long range of >8 hours for the compound of Formula I. Consequently, rapid hepatic clearance is unlikely to contribute to low oral bioavailability.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,958,458; 5,972,891; 5,980,945; 5,993,855; 6,045,830; 6,087,324; 6,113,943; 6,197,350; 6,248,363; 6,264,970; 6,267,981; 6,270,798; 6,375,987; 6,376,461; 6,419,961; 6,589,548; 6,613,358; 6,623,756; 6,699,500; 6,793,936; 6,827,947; 6,902,742; 6,958,161; 7,255,876; 7,416,738; 7,427,414; 7,485,322; Bussemer et al., Crit. Rev. Ther. Drug Carrier Syst. 2001, 18, 433-458; Modified-Release Drug Delivery Technology, 2nd ed.; Rathbone et al., Eds.; Marcel Dekker AG: 2005; Maroni et al., Expert. Opin. Drug Deliv. 2005, 2, 855-871; Shi et al., Expert Opin. Drug Deliv. 2005, 2, 1039-1058; Polymers in Drug Delivery; Ijeoma et al., Eds.; CRC Press LLC: Boca Raton, Fla., 2006; Badawy et al., J. Pharm. Sci. 2007, 9, 948-959; Modified-Release Drug Delivery Technology, supra; Conway, Recent Pat. Drug Deliv. Formul. 2008, 2, 1-8; Gazzaniga et al., Eur. J. Pharm. Biopharm. 2008, 68, 11-18; Nagarwal et al., Curr. Drug Deliv. 2008, 5, 282-289; Gallardo et al., Pharm. Dev. Technol. 2008, 13, 413-423; Chrzanowski, AAPS PharmSciTech. 2008, 9, 635-638; Chrzanowski, AAPS PharmSciTech. 2008, 9, 639-645; Kalantzi et al., Recent Pat. Drug Deliv. Formul. 2009, 3, 49-63; Saigal et al., Recent Pat. Drug Deliv. Formul. 2009, 3, 64-70; and Roy et al., J. Control Release 2009, 134, 74-80.

### 1. Matrix Controlled Release Devices

The pharmaceutical compositions provided herein in a modified release dosage form can be fabricated using a matrix controlled release device known to those skilled in the art. See, Takada et al. in Encyclopedia of Controlled Drug Delivery; Mathiowitz Ed.; Wiley: 1999; Vol 2.

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, D E) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, Remington: The Science and Practice of Pharmacy, supra; Santus and Baker, J. Controlled Release 1995, 35, 1-21; Verma et al., Drug Development and Industrial Pharmacy 2000, 26, 695-708; and Verma et al., J. Controlled Release 2002, 79, 7-27.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and International Pat. Appl. Publ. No. WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Ghebre-Sellassie Ed.; Marcel Dekker: 1994; and Pharmaceutical Pelletization Technology; Ghebre-Sellassie Ed.; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of a subject to be treated, including liposome-, resealed erythrocyte-, and antibody-based delivery systems. Examples include, but are not limited to, those disclosed in U.S. Pat. Nos. 5,709,874; 5,759,542; 5,840,674; 5,900,252; 5,972,366; 5,985,307; 6,004,534; 6,039,975; 6,048,736; 6,060,082; 6,071,495; 6,120,751; 6,131,570; 6,139,865; 6,253,872; 6,271,359; 6,274,552; 6,316,652; and 7,169,410.

### Methods of Use

In one embodiment, provided herein are methods for treating or preventing a Flaviviridae, including hepatitis C, viral infection in a subject, which comprises administering to a subject a therapeutically effective amount of a crystalline or salt form of the compound of Formula I, or a pharmaceutically acceptable solvate or prodrug thereof. In one embodiment, the subject is a mammal. In another embodiment, the subject is a human.

In one embodiment, the Flaviviridae viral infection is caused by hepatitis C virus. In one embodiment, the Flaviviridae viral infection is caused by Yellow Fever virus. In one embodiment, the Flaviviridae viral infection is caused by West Nile virus. In one embodiment, the Flaviviridae viral infection is caused by dengue virus. In one embodiment, the Flaviviridae viral infection is caused by GV virus A. In one embodiment, the Flaviviridae viral infection is caused by GB virus C. In one embodiment, the Flaviviridae viral infection is caused by GB virus D. In one embodiment, the Flaviviridae viral infection is caused by bovine viral diarrhea virus 1. In one embodiment, the Flaviviridae viral infection is caused by bovine viral diarrhea virus 2. In one embodiment, the Flaviviridae viral infection is caused by classical swine fever virus. In one embodiment, the Flaviviridae viral infection is caused by Japanese encephalitis virus. In one embodiment, the Flaviviridae viral infection is caused by Saint Louis encephalitis virus. In one embodiment, the Flaviviridae viral infection is caused by tick-borne encephalitis. In one embodiment, the Flaviviridae viral infection is caused by Murray Valley encephalitis virus. In one embodiment, the Flaviviridae viral infection is caused by Kyasanur Forest disease virus. In one embodiment, the Flaviviridae viral infection is caused by Omsk haemorrhagic fever virus. In one embodiment, the Flaviviridae viral infection is caused by Aedes flavivirus. In one embodiment, the Flaviviridae viral infection is caused by Apoi virus. In one embodiment, the Flaviviridae viral infection is caused by Aroa virus. In one embodiment, the Flaviviridae viral infection is caused by Bagaza virus. In one embodiment, the Flaviviridae viral infection is caused by Banzi virus. In one embodiment, the Flaviviridae viral infection is caused by Barkedji virus. In one embodiment, the Flaviviridae viral infection is caused by Bouboui virus. In one embodiment, the Flaviviridae viral infection is caused by Bussuquara virus. In one embodiment, the Flaviviridae viral infection is caused by Bukalasa bat virus. In one embodiment, the Flaviviridae viral infection is caused by Cacipacore virus. In one embodiment, the Flaviviridae viral infection is caused by Calbertado virus. In one embodiment, the Flaviviridae viral infection is caused by Carey Island virus. In one embodiment, the Flaviviridae viral infection is caused by Cell fusing agent virus. In one embodiment, the Flaviviridae viral infection is caused by Chaoyang virus. In one embodiment, the Flaviviridae viral infection is caused by Cowbone Ridge virus. In one embodiment, the Flaviviridae viral infection is caused by Culex flavivirus. In one embodiment, the Flaviviridae viral infection is caused by Culex theileri flavivirus. In one embodiment, the Flaviviridae viral infection is caused by Dakar bat virus. In one embodiment, the Flaviviridae viral infection is caused by Deer tick virus. In one embodiment, the Flaviviridae viral infection is caused by Donggang virus. In one embodiment, the Flaviviridae viral infection is caused by Duck egg drop syndrome virus. In one embodiment, the Flaviviridae viral infection is caused by Edge Hill virus. In one embodiment, the Flaviviridae viral infection is caused by Entebbe bat virus. In one embodiment, the Flaviviridae viral infection is caused by Gadgets Gully virus. In one embodiment, the Flaviviridae viral infection is caused by Ilheus virus. In one embodiment, the Flaviviridae viral infection is caused by Israel turkey meningoencephalomyelitis virus. In one embodiment, the Flaviviridae viral infection is caused by Jugra virus. In one embodiment, the Flaviviridae viral infection is caused by Jutiapa virus. In one embodiment, the Flaviviridae viral infection is caused by Kadam virus. In one embodiment, the Flaviviridae viral infection is caused by Kamiti River virus. In one embodiment, the Flaviviridae viral infection is caused by Karshi virus. In one embodiment, the Flaviviridae viral infection is caused by Kedougou virus. In one embodiment, the Flaviviridae viral infection is caused by Kokobera virus. In one embodiment, the Flaviviridae viral infection is caused by Koutango virus. In one embodiment, the Flaviviridae viral infection is caused by Kyasanur forest disease virus. In one embodiment, the Flaviviridae viral infection is caused by Lammi virus. In one embodiment, the Flaviviridae viral infection is caused by Langat virus. In one embodiment, the Flaviviridae viral infection is caused by Louping ill virus. In one embodiment, the Flaviviridae viral infection is caused by Meaban virus. In one embodiment, the Flaviviridae viral infection is caused by Modoc virus. In one embodiment, the Flaviviridae viral infection is caused by Montana myotis leukoencephalitis virus. In one embodiment, the Flaviviridae viral infection is caused by Nakiwogo virus. In one embodiment, the Flaviviridae viral infection is caused by Nounane virus. In one embodiment, the Flaviviridae viral infection is caused by Ntaya virus. In one embodiment, the Flaviviridae viral infection is caused by Phnom Penh bat virus. In one embodiment, the Flaviviridae viral infection is caused by Pan virus. In one embodiment, the Flaviviridae viral infection is caused by Quang Binh virus. In one embodiment, the Flaviviridae viral infection is caused by Rio Bravo virus. In one embodiment, the Flaviviridae viral infection is caused by Rocio virus. In one embodiment, the Flaviviridae viral infection is caused by Royal Farm virus. In one embodiment, the Flaviviridae viral infection is caused by Saboya virus. In one embodiment, the Flaviviridae viral infection is caused by Sal Vieja virus. In one embodiment, the Flaviviridae viral infection is caused by San Perlita virus. In one embodiment, the Flaviviridae viral infection is caused by Saumarez Reef virus. In one embodiment, the Flaviviridae viral infection is caused by Sepik virus. In one embodiment, the Flaviviridae viral infection is caused by Tembusu virus. In one embodiment, the Flaviviridae viral infection is caused by Turkish sheep encephalitis virus. In one embodiment, the Flaviviridae viral infection is caused by Tyuleniy virus. In one embodiment, the Flaviviridae viral infection is caused by Uganda S virus. In one embodiment, the Flaviviridae viral infection is caused by Usutu virus. In one embodiment, the Flaviviridae viral infection is caused by Wesselsbron virus. In one embodiment, the Flaviviridae viral infection is caused by Yaounde virus. In one embodiment, the Flaviviridae viral infection is caused by Yokose virus. In one embodiment, the Flaviviridae viral infection is caused by Zika virus. In one embodiment, the Flaviviridae viral infection is caused by Border disease virus.

As used herein, the term “hepatitis C virus” or “HCV” refers to a viral species or a genetic variation thereof, a pathogenic strain of which causes hepatitis C. Examples of HCV include, but are not limited to, HCV genotypes 1, 2, 3, 4, and 5, and subtype 1a, 1b, 1c, 2a, 2b, 2c, 3a, and 3b.

In one embodiment, the hepatitis C viral infection is caused by HCV genotype 1. In certain embodiments, the hepatitis C viral infection is caused by HCV subtype 1a. In certain embodiments, the hepatitis C viral infection is caused by HCV subtype 1b. In certain embodiments, the hepatitis C viral infection is caused by HCV subtype 1c. In another embodiment, the hepatitis C viral infection is caused by HCV genotype 2. In certain embodiments, the hepatitis C viral infection is caused by HCV subtype 2a. In certain embodiments, the hepatitis C viral infection is caused by HCV subtype 2b. In certain embodiments, the hepatitis C viral infection is caused by HCV subtype 2c. In yet another embodiment, the hepatitis C viral infection is caused by HCV genotype 3. In certain embodiments, the hepatitis C viral infection is caused by HCV subtype 3a. In certain embodiments, the hepatitis C viral infection is caused by HCV subtype 3b. In yet another embodiment, the hepatitis C viral infection is caused by HCV genotype 4. In yet another embodiment, the hepatitis C viral infection is caused by HCV genotype 5. In yet another embodiment, the hepatitis C viral infection is caused by HCV genotype 6.

In another embodiment, provided herein is a method for inhibiting replication of a virus in a subject, which comprises contacting a subject with a therapeutically effective amount of a crystalline or salt form of the compound of Formula I, or an isotopic variant thereof; or a pharmaceutically acceptable solvate or prodrug thereof. In one embodiment, the subject is a cell. In another embodiment, the subject is a human cell. In yet another embodiment, the subject is a mammal. In still another embodiment, the subject is human.

In one embodiment, the virus is a hepatitis C virus. In another embodiment, the virus is HCV genotype 1. In certain embodiments, the virus is HCV subtype 1a. In yet certain embodiments, the virus is HCV subtype 1b. In certain embodiments, the virus is HCV subtype 1c. In another embodiment, the virus is HCV genotype 2. In certain embodiments, the virus is HCV subtype 2a. In certain embodiments, the virus is HCV subtype 2b. In certain embodiments, the virus is HCV subtype 2c. In yet another embodiment, the virus is HCV genotype 3. In certain embodiments, the virus is HCV subtype 3a. In certain embodiments, the virus is HCV subtype 3b. In yet another embodiment, the virus is HCV genotype 4. In yet another embodiment, the virus is HCV genotype 5. In yet another embodiment, the virus is HCV genotype 6.

In certain embodiments, administration of a therapeutically effective amount of a crystalline or salt form of the compound of Formula I, or an isotopic variant thereof; or a pharmaceutically acceptable solvate or prodrug thereof results in a 10%, 20%, 30%, 40%, 50%, 60%, 70%, 80%, 90%, 95%, 99% or more reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer.

In certain embodiments, administration of a therapeutically effective amount of a crystalline or salt form of the compound of Formula I, or an isotopic variant thereof; or a pharmaceutically acceptable solvate or prodrug thereof results in a 1, 2, 3, 4, 5, 10, 15, 20, 25, 50, 75, 100-fold or more reduction in the replication of the virus relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a crystalline or salt form of the compound of Formula I, or an isotopic variant thereof; or a pharmaceutically acceptable solvate or prodrug thereof results in a 10%, 20%, 30%, 40%, 50%, 60%, 70%, 80%, 90%, 95%, 99% or more reduction in the viral titer relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a crystalline or salt form of the compound of Formula I, or an isotopic variant thereof; or a pharmaceutically acceptable solvate or prodrug thereof results in a 1, 2, 3, 4, 5, 10, 15, 20, 25, 50, 75, 100 or more fold reduction in the viral titer relative to a subject without administration of the compound, as determined at 1 day, 2 days, 3 days, 4 days, 5 days, 10 days, 15 days, or 30 days after the administration by a method known in the art.

In yet another embodiment, provided herein is a method for inhibiting the replication of an HCV virus, which comprises contacting the virus with a therapeutically effective amount of a crystalline or salt form of the compound of Formula I, or an isotopic variant thereof; or a pharmaceutically acceptable solvate or prodrug thereof.

In one embodiment, the HCV is genotype 1. In certain embodiments, the HCV is subtype 1a. In certain embodiments, the HCV is subtype 1b. In certain embodiments, the HCV is subtype 1c. In another embodiment, the HCV is genotype 2. In certain embodiments, the HCV is subtype 2a. In certain embodiments, the HCV is subtype 2b. In certain embodiments, the HCV is subtype 2c. In yet another embodiment, the HCV is genotype 3. In certain embodiments, the HCV is subtype 3a. In certain embodiments, the HCV is subtype 3b. In yet another embodiment, the HCV is genotype 4. In yet another embodiment, the HCV is genotype 5. In yet another embodiment, the HCV is genotype 6.

In certain embodiments, the contacting of the virus with a therapeutically effective amount of a crystalline or salt form of the compound of Formula I, or an isotopic variant thereof; or a pharmaceutically acceptable solvate or prodrug thereof results in a 10%, 20%, 30%, 40%, 50%, 60%, 70%, 80%, 90%, 95%, 99% or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of a crystalline or salt form of the compound of Formula I, or an isotopic variant thereof; or a pharmaceutically acceptable solvate or prodrug thereof results in a 1, 2, 3, 4, 5, 10, 15, 20, 25, 50, 75, 100 or more fold reduction in the viral titer relative to the virus without such contact, as determined at 1 day, 2 days, 3 days, 4 days, 5 days, 10 days, 15 days, or 30 days after the initial contact, by a method known in the art.

In still another embodiment, provided herein is a method for treating, preventing, or ameliorating one or more symptoms of a liver disease or disorder associated with an HCV infection, comprising administering to a subject a therapeutically effective amount of a crystalline or salt form of the compound of Formula I, or an isotopic variant thereof; or a pharmaceutically acceptable solvate or prodrug thereof. Non-limiting examples of diseases associated with HCV infection include chronic hepatitis, cirrhosis, hepatocarcinoma, or extra hepatic manifestation.

Depending on the condition, disorder, or disease, to be treated and the subject's condition, a compound provided herein may be administered by oral, parenteral (e.g., intramuscular, intraperitoneal, intravenous, intracerebroventricular (ICV), intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1,000 milligram, from about 0.1 to about 500 milligrams, or from 0.5 about to about 100 milligram active ingredient(s) per dosage unit, and if the condition of the patient requires, the dose can, by way of alternative, be administered as a continuous infusion.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the subject undergoing therapy.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds provided herein may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to prevent, treat, or manage a condition, disorder, or disease, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a condition, disorder, or disease. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, treatment, or management of a condition, disorder, or disease). In addition, a synergistic effect can result in improved efficacy of agents in the prevention, treatment, or management of a condition, disorder, or disease. Finally, a synergistic effect of a combination of therapies (e.g., a combination of prophylactic or therapeutic agents) may avoid or reduce adverse or unwanted side effects associated with the use of either therapy alone.

The compound provided herein can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by a pharmaceutical composition comprising a crystalline or salt form of the compound of Formula I, or a pharmaceutically acceptable solvate or prodrug thereof, provided herein, in combination or alternation with a second, and perhaps third, antiviral compound that induces a different mutation from that caused by the principal drug. Alternatively, the pharmacokinetics, biodistribution, or other parameters of the drug can be altered by such combination or alternation therapy. In general, combination therapy is typically preferred over alternation therapy because it induces multiple simultaneous stresses on the virus.

In one embodiment, a pharmaceutical composition comprises a crystalline or salt form of the compound of Formula I and one or more pharmaceutically acceptable carriers. In one embodiment, a pharmaceutical composition further comprises a second antiviral agent. In one embodiment, the second antiviral agent is selected from an interferon, ribavirin, amantadine, an interleukin, an NS3 protease inhibitor, an NS5A inhibitor, an NS5B inhibitor, a cyclophilin inhibitor, a cysteine protease inhibitor, a phenanthrenequinone, a thiazolidine, a benzanilide, a helicase inhibitor, a polymerase inhibitor, a nucleotide analogue, a nucleoside analogue, a gliotoxin, a cerulenin, an antisense phosphorothioate oligodeoxynucleotide, an inhibitor of IRES-dependent translation, or a ribozyme. In one embodiment, the second antiviral agent is an interferon. In another embodiment, the interferon is selected from pegylated interferon alpha 2a, interferon alfacon-1, natural interferon, ALBUFERON®, interferon beta-1a, omega interferon, interferon alpha, interferon gamma, interferon tau, interferon delta, or interferon gamma-1b.

In certain embodiments, a pharmaceutical composition comprising a crystalline or salt form of the compound of Formula I, or a pharmaceutically acceptable solvate or prodrug thereof, is combined with a cyclophilin inhibitor, including, but not limited to, alisporivir (Novartis), cyclosporin A, sanglifehrins and sanglifehrin analogs, CsD, NIM-811, and SCY-635.

In certain embodiments, a pharmaceutical composition comprising a crystalline or salt form of the compound of Formula I, or a pharmaceutically acceptable solvate or prodrug thereof, is combined with an NS5A inhibitor, including, but not limited to, ABT-267, BMS-790052, GS-5885, GS-5816, PPI-461, and PPI-668.

In certain embodiments, a pharmaceutical composition comprising a crystalline or salt form of the compound of Formula I, or a pharmaceutically acceptable solvate or prodrug thereof, is combined with an NS5B inhibitor, including, but not limited to, ABT-072, ABT-333, ANA598, BI 207127, GS-9669, GS-9190, GSK-625433, HCV-796, IDX184, IDX375, IDX19368, JTK-109, MK-0608, MK-3281, NM283, PF-868554, PSI-879, PSI-938, PSI-6130, PSI-7851, PSI-7977, R1626, R7128, VCH-222, VCH-759, and VCH-916. In one embodiment, the NS5B inhibitor is PSI-7977 (sofosbuvir). In another embodiment, the NS5B inhibitor is one or more NS5B inhibitors described in U.S. Patent Publication No. 2013-0315868, hereby incorporated by reference herein, it its entirety.

In certain embodiments, a pharmaceutical composition comprising a crystalline or salt form of the compound of Formula I, or a pharmaceutically acceptable solvate or prodrug thereof, is combined with an HCV protease inhibitor, including, but not limited to, ACH-0141625, faldaprevir (BI 201335); asunaprevir (BMS-650032); TMC 435 or TMC 435350 (Medivir/Tibotec); ITMN 191/R7227 (InterMune); MK 7009 (Merck); SCH 5034/SCH 503034/Boceprevir and SCH 900518/narlaprevir (Schering); VX950/telaprevir (Vertex); substrate-based NS3 protease inhibitors (DE 19914474, WO 98/17679, WO 98/22496, WO 99/07734, and Attwood et al., Antiviral Chemistry and Chemotherapy 1999, 10, 259-273); non-substrate-based NS3 protease inhibitors, including 2,4,6-trihydroxy-3-nitro-benzamide derivatives (Sudo et al., Biochem. Biophys. Res. Commun. 1997, 238, 643-647), a phenanthrenequinone (Chu et al., Tetrahedron Letters 1996, 37, 7229-7232), RD3-4082, RD3-4078, SCH 68631, and SCH 351633 (Chu et al., Bioorganic and Medicinal Chemistry Letters 1999, 9, 1949-1952); and Eglin C, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinás-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,608,027; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; and 7,491,794; U.S. Pat. App. Pub. Nos.: 2002/0016294, 2002/0016442; 2002/0032175; 2002/0037998; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781, 2008/0152622, 2009/0035271, 2009/0035272, 2009/0047244, 2009/0111969, 2009/0111982, 2009/0123425, 2009/0130059, 2009/0148407, 2009/0156800, 2009/0169510, 2009/0175822, 2009/0180981, and 2009/0202480; U.S. patent application Ser. No. 12/365,127; and International Pat. App. Pub. Nos.: WO 98/17679; WO 98/22496; WO 99/07734; WO 00/09543; WO 00/59929; WO 02/08187; WO 02/08251; WO 02/08256; WO 02/08198; WO 02/48116; WO 02/48157; WO 02/48172; WO 02/60926; WO 03/53349; WO 03/64416; WO 03/64455; WO 03/64456; WO 03/66103; WO 03/99274; WO 03/99316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; WO 2007/015824, WO 2007/056120, WO 2008/019289, WO 2008/021960, WO 2008/022006, WO 2008/086161, WO 2009/053828, WO 2009/058856, WO 2009/073713, WO 2009/073780, WO 2009/080542, WO 2009/082701, WO 2009/082697, and WO 2009/085978; the disclosure of each of which is incorporated herein by reference in its entirety.

Other protease inhibitors include thiazolidine derivatives, such as RD-1-6250, RD4 6205, and RD4 6193 (Sudo et al., Antiviral Research 1996, 32, 9-18); and thiazolidines and benzanilides (Kakiuchi et al., FEBS Lett. 1998, 421, 217-220; and Takeshita et al., Analytical Biochemistry 1997, 247, 242-246).

Suitable helicase inhibitors include, but are not limited to, those disclosed in U.S. Pat. No. 5,633,358; and International Pat. App. Pub. No. WO 97/36554.

Suitable nucleotide polymerase inhibitors include, but are not limited to, 2′-methyl ribofuranosyl nucleotides. See, e.g., WO 01/90121, WO 01/92282, WO 2004/002999, WO 2005/003147, U.S. Pat. Nos. 6,914,054; 7,608,597; 7,608,600; 7,824,851; 7,157,441; 7,635,689; 7,429,572; 7,754,699; 7,964,580; 7,105,499; 6,777,395; 8,481,712. In one embodiment, a nucleotide polymerase inhibitor is gliotoxin (Ferrari et al., Journal of Virology 1999, 73, 1649-1654), cerulenin (Lohmann et al., Virology 1998, 249, 108-118), ABT-072, ABT-333, AG-02154, ANA598, ANA773, GS-9190, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, R7128, RG7128, VCH-759, VCH-916 or VX-222 (VCH-222).

Suitable interfering RNA (iRNA) based antivirals include, but are not limited to, short interfering RNA (siRNA) based antivirals, such as Sirna-034 and those described in International Pat. App. Pub. Nos. WO/03/070750 and WO 2005/012525, and U.S. Pat. App. Pub. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; and Galderisi et al., Journal of Cellular Physiology 1999, 181, 251-257);

Suitable inhibitors of IRES-dependent translation include, but are not limited to, those described in Japanese Pat. App. Pub. Nos.: JP 08268890 and JP 10101591.

Suitable ribozymes include those disclosed in, for example, U.S. Pat. Nos. 6,043,077; 5,869,253; and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. App. Pub. Nos. 2004/0121980; 2005/0009737; 2005/0038240; and 2006/0040890; and International Pat. App. Pub. Nos: WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, INTRON® A (interferon alfa-2b), PEGASYS® (Peginterferon alfa-2a) ROFERON® A (recombinant interferon alfa-2a), INFERGEN® (interferon alfacon-1), and PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, one or more compounds provided herein are administered in combination or alternation with an anti-hepatitis C virus polymerase inhibitor, such as ribavirin, viramidine, NM 283 (valopicitabine), ABT-072, ABT-267, ABT-333, AG-02154, ANA598, ANA773, EDP-239, deleobuvir (BI 207127), GS-9190, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, HCV-796, R7128, RG7128, VCH-759, VCH-916, VX-222 (VCH-222), and those as disclosed in U.S. Pat. Appl. Publ. Nos. 2009/0081158 and 2009/0238790, the disclosure of each of which is incorporated herein by reference in its entirety.

In certain embodiments, the one or more compounds provided herein are administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), and PEG-INTRON® (pegylated interferon alfa-2b).

In certain embodiments, one or more compounds provided herein are administered in combination or alternation with an anti-hepatitis C virus protease inhibitor, such as ABT-450, ITMN-191, SCH 503034, VX950 (telaprevir), and TMC 435.

In certain embodiments, one or more compounds provided herein are administered in combination or alternation with an anti-hepatitis C virus vaccine, including, but not limited to, TG4040, PEVIPRO™, CGI-5005, HCV/MF59, GV1001, IC41, and INNO0101 (E1).

In certain embodiments, one or more compounds provided herein are administered in combination or alternation with an anti-hepatitis C virus monoclonal antibody, such as AB68 and XTL-6865 (formerly HepX-C); or an anti-hepatitis C virus polyclonal antibody, such as cicavir.

In certain embodiments, one or more compounds provided herein are administered in combination or alternation with an anti-hepatitis C virus immunomodulator, such as ZADAXIN® (thymalfasin), NOV-205, and oglufanide.

In certain embodiments, one or more compounds provided herein are administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide), and PYN17.

The compounds provided herein can also be administered in combination with other classes of compounds, including, but not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) farnesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a crystalline or salt form of the compound of Formula I, or an isotopic variant thereof; or a pharmaceutically acceptable solvate or prodrug thereof.

In certain embodiments, the kit includes a container comprising a dosage form of a crystalline or salt form of the compound of Formula I, or an isotopic variant thereof; or a pharmaceutically acceptable solvate or prodrug thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); L, (liter); mM (millimolar); μM (micromolar); Hz (Hertz); MHz (megahertz); mmol (millimoles); eq. (equivalent); hr or hrs (hours); min (minutes); MS (mass spectrometry); NMR (nuclear magnetic resonance); HPLC, (high-performance liquid chromatography or high pressure liquid chromatography); DCM (dichloromethane); DMF (N,N-dimethylformamide); DMSO (dimethylsulfoxide); DMSO-d₆ (deuterated dimethylsulfoxide); EtOAc (ethyl acetate); Et₂O (diethyl ether); EtOH (ethanol); IPA (isopropyl alcohol); IPAc (isopropyl acetate); MEK (methyl ethyl ketone); MeOH (methanol); MTBE (methyl t-butyl ether); PE (petroleum ether); THF (tetrahydrofuran); DIPE (diisopropyl ether); DIPEA (N,N-diisopropylethylamine); TEA (triethylamine); TFA (trifluoroacetic acid); Me (methyl); Et (ethyl); iPr, (isopropyl); tBu (tert-butyl); Bn (benzyl); Ph (phenyl); AcO (acetate); X-ray powder diffractometry (XRPD); differential scanning calorimetry (DSC); modulated DCS (mDCS); microscopy; ion chromatography (IC); thermogravimetry (TGA); and gravimetric vapor sorption (GVS).

### Example 1

### General Characterization Methods of Form A

The crystals of crystalline Form A of the compound of Formula I were characterized by bright field and cross-polarized optical microscopy (PLM), Karl Fischer titration, thermodynamic aqueous solubility, XRPD, DSC, TGA, GVS, and HPLC.

X-Ray Powder Diffraction patterns were collected on a Bruker D8 diffractometer using Cu Ka radiation (40 kV, 40 mÅ), θ-2θ goniometer, and divergence of V4 and receiving slits, a Ge monochromator and a Lynxeye detector. The instrument was performance checked using a certified Corundum standard (NIST 1976). The software used for data collection was Diffrac Plus XRD Commander v2.5.0 and the data were analyzed and presented using Diffrac Plus EVA v11.0.0.2 or v13.0.0.2. Samples were run under ambient conditions as flat plate specimens using powder as received. The sample was gently packed into a cavity cut into polished, zero-background (510) silicon wafer. The sample was rotated in its own plane during analysis. The details of the data collection are: angular range: 2 to 42° 2θ; step size: 0.05° 2θ; and collection time: 0.5 s/step.

Differential scanning calorimetry data were collected on a TA Instruments Q2000 equipped with a 50 position autosampler. The calibration for thermal capacity was carried out using sapphire and the calibration for energy and temperature was carried out using certified indium. Typically 0.5-3 mg of each sample, in a pin-holed aluminium pan, was heated at 10° C./min from 25° C. to 300° C. A purge of dry nitrogen at 50 mL/min was maintained over the sample. Modulated temperature DSC was carried out using an underlying heating rate of 2° C./min and temperature modulation parameters of ±0.318° C. (amplitude) every 60 seconds (period). The instrument control software was Advantage for Q Series v2.8.0.392 and Thermal Advantage v4.8.3 and the data were analyzed using Universal Analysis v4.4A.

Amorphous compounds were analyzed by modulated DSC (mDSC) to attempt to detect the glass transition temperature (Tg), as this transition was unresolved in standard DSC. Samples of 3-5 mg of amorphous material was loaded into aluminum sample pans, which were then hermetically sealed. The modulation amplitude was ±0.5° C., and the modulation frequency was 40 seconds. The samples were analyzed over the temperature range of ambient to 250° C. in modulated mode with a 5° C./min ramp rate. The deconvoluted thermograms showed total heat flow and its reversing and non-reversing components. The compound of Formula I has an apparent glass transition region in the range of 133-148° C., with a T_{g} of approximately 140° C.

Qualitative assessments of particle size and shape were obtained by bright field microscopy. Cross-polarized microscopy was used to assess the particles for indications of crystallinity by monitoring birefringence. The microscopy was performed on powder and smeared powder samples, and the results were recorded using a Zeiss Axioskop 40 Pol microscope in conjunction with a Zeiss MRc5 digital camera. A sample of the compound of Formula I was placed on a microscope slide. The material appears glassy by bright field and cross-polarized microscopy. The particle and/or agglomerate sizes of each vary greatly, from a few microns to several hundred. In the smeared samples, the apparent glassy character is more pronounced, as several of the smeared portions appear transparent under bright light and are not birefringent under cross-polarized light. Portions of each sample appear birefringent under cross-polarized light; however, this is an artifact of the thickness of the particulates rather than an indication of crystallinity.

The water content of each sample was measured by Karl Fischer titration on a Metrohm 874 Oven Sample Processor at 150° C. with 851 Titrano Coulometer using Hydranal Coulomat AG oven reagent and nitrogen purge. Weighed solid samples were introduced into a sealed sample vial. Approx 10 mg of sample was used per titration and duplicate determinations were made. The water content of Form A of the compound of Formula I was found to be 0.24% by weight.

Aqueous solubility was determined by suspending sufficient compound in water to give a maximum final concentration of ≧10 mg/mL of the parent free-form of the compound. The suspension was equilibrated at 25° C. for 24 hours then the pH was measured. The suspension was then filtered through a glass fibre C filter. The filtrate was then diluted by an appropriate factor, e.g. 101. Quantitation was by HPLC with reference to a standard solution of approximately 0.25 mg/mL in DMSO. Different volumes of the standard, diluted and undiluted sample solutions were injected. The solubility was calculated using the peak areas determined by integration of the peak found at the same retention time as the principal peak in the standard injection. On a duplicate run, the aqueous solubility of Form A of the compound of Formula I was found to be 0.03 mg/mL and 0.01 mg/mL (pH 6.42).

The thermogravimetric analysis data were collected on a TA Instruments Q500 TGA, equipped with a 16 position autosampler. The instrument was temperature calibrated using certified Alumel and Nickel. Typically 5-10 mg of each sample was loaded onto a pre-tared aluminium DSC pan and heated at 10° C./min from ambient temperature to 350° C. A nitrogen purge at 60 mL/min was maintained over the sample. The instrument control software was Advantage for Q Series v2.8.0.392 and Thermal Advantage v4.8.3 and the data were analyzed using Universal Analysis v4.4A.

Sorption isotherms were obtained using a SMS DVS Intrinsic moisture sorption analyzer, controlled by DVS Intrinsic Control software v1.0.0.30. The sample temperature was maintained at 25° C. by the instrument controls. The humidity was controlled by mixing streams of dry and wet nitrogen, with a total flow rate of 200 mL/min. The relative humidity (RH) was measured by a calibrated Rotronic probe (dynamic range of 1.0-100% RH), located near the sample. The weight change, (mass relaxation) of the sample as a function of % RH was constantly monitored by the microbalance (accuracy ±0.005 mg). Typically 5-20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0-90% RH range. Data analysis was undertaken in Microsoft Excel using DVS Analysis Suite v6.0.0.7.

Ion chromatography data were collected on a Metrohm 861 Advanced Compact IC (for anions) using IC Net software v2.3. Accurately weighed samples were prepared as stock solutions in an appropriate dissolving solution and diluted appropriately prior to testing. Eluent was 3.2 mM sodium carbonate or 1.0 mM sodium bicarbonate in 5% aqueous acetone. Quantification was achieved by comparison with standard solutions of known concentration of the ion being analyzed.

HPLC analysis was performed on an Agilent HP1100/1200 system equipped with a diode array detector and using Chemstation software. Test samples were dissolved to approximately 0.1 mg/mL in MeOH (injection volume of 8 μL), the flow rate was 2.5 mL/min and the detection wavelengths were 254 and 272 nm.

The X-ray powder diffraction pattern of the crystals of Form A of the compound of Formula I is illustrated in FIG. 1. The crystals of crystal Form A of the compound of Formula I have characteristic XRP diffraction peaks expressed in two-theta at approximately 2.7, 8.1, 11.4, 12.5, 15.4, 16.3, 16.8, 19.7, 20.6, 21.5, 22.7, 23.5, 23.7, and 25.1° (Table 1).

TABLE 1
X-Ray Powder Diffraction Peaks for Crystal Form A

| Angle | |
|---|---|
| 2θ° | Intensity % |
| 2.67 | 100.0 |
| 8.11 | 30.6 |
| 10.48 | 7.4 |
| 10.69 | 5.1 |
| 11.40 | 26.6 |
| 11.73 | 10.2 |
| 12.04 | 6.0 |
| 12.51 | 29.6 |
| 13.20 | 7.9 |
| 13.93 | 12.5 |
| 14.74 | 7.2 |
| 15.40 | 64.0 |
| 16.34 | 83.5 |
| 16.80 | 38.5 |
| 17.40 | 8.3 |
| 18.21 | 15.2 |
| 18.89 | 11.0 |
| 19.68 | 31.3 |
| 20.56 | 26.3 |
| 21.50 | 29.3 |
| 22.74 | 37.2 |
| 23.49 | 26.0 |
| 23.74 | 24.5 |
| 24.20 | 15.9 |
| 24.59 | 10.3 |
| 25.07 | 38.8 |
| 25.64 | 16.2 |
| 26.81 | 16.0 |
| 27.38 | 10.8 |
| 27.99 | 10.8 |
| 28.19 | 10.7 |
| 28.57 | 6.0 |
| 29.06 | 5.8 |
| 29.24 | 6.4 |
| 29.77 | 4.5 |

The differential scanning calorimetric thermogram of the crystals of Form A of the compound of Formula I revealed the crystals exhibit an endotherm with a peak temperature of 253° C. and an onset temperature of 247° C.

The thermogravimetric analysis thermogram of the crystals of Form A of the compound of Formula I revealed the crystals exhibit a weight loss of less than 1.7% and less than 3% between 90-225° C., and 25-250° C., respectively.

### Example 2

### Initial Polymorphism Study

Amorphous form of the compound of Formula I (ca. 10 mg) was weighed into twenty-four amber vials and five volumes of solvent (50 μL) added at ambient temperature. The vials were then left at ambient conditions for thirty minutes. If the material had not dissolved in this time, the samples were placed at 50° C. in a shaker for one hour. After this time at the elevated temperature, if the material had not dissolved, the process was repeated with additional volumes of solvent; totaling five, ten fifteen, twenty-five and fifty volumes. If solutions were obtained no additional solvent was added unless the sample was observed to precipitate out in which case solvent addition was continued as previously. Samples which were slurries were placed in a maturation chamber (cycling between 50° C. and ambient for four hours each) over four days. Solutions were placed at room temperature and the solvent was allowed to evaporate slowly through a pin-hole. Aliquots of material were removed by pipette, filtered under vacuum and analyzed by XRPD. The results are summarized in Table 2.

TABLE 2
Initial Polymorphism Screen

| | 5 vol | | 10 vol | | 15 vol | | 25 vol | | 50 vol | | | |
|---|---|---|---|---|---|---|---|---|---|---|---|---|
| Solvent | RT | 50° C. | RT | 50° C. | RT | 50° C. | RT | 50° C. | RT | 50° C. | Technique | XRPD |
| Heptane | x | X | x | x | x | x | x | x | x | x | Maturation | Amorphous |
| Dioxane | x | ✓ | — | — | — | — | — | — | ✓ | | Maturation | Oil |
| Toluene | x | x | x | x | x | x | x | x | x | x | Maturation | Form A |
| Transcutol | x | x | x | x | x | x | x | x | x | x | Maturation | Low cryst. |
| | | | | | | | | | | | | Form A |
| MTBE | x | x | x | x | x | x | x | x | x | x | Maturation | Form A |
| Tetraline | x | x | x | x | x | x | x | x | x | x | Maturation | Amorphous |
| DIPE | x | x | x | x | x | x | x | x | x | x | Maturation | Low cryst. |
| | | | | | | | | | | | | Form A |
| Anisole | x | ✓ | — | — | — | — | — | — | — | — | Maturation | Form A |
| EtOAc | x | x | x | x | x | x | x | x | x | x | Maturation | Form A |
| IPAc | x | x | x | x | x | x | x | x | x | x | Maturation | Form A |
| IPA | x | x | x | x | x | x | x | x | x | x | Maturation | Form A |
| THF | x | x | x | x | x | x | x | x | x | x | Maturation | Form A |
| DCM | ✓ | ✓ | — | — | — | — | — | — | ✓ | | Slow evap. | Amorphous |
| MEK | ✓ | — | — | — | — | — | — | — | — | — | Maturation | Form A + |
| | | | | | | | | | | | | additional |
| | | | | | | | | | | | | peak |
| Acetone | ✓ | — | — | — | — | — | — | — | — | — | Maturation | Form A |
| Methanol | ✓ | ✓ | — | — | — | — | — | — | — | — | Maturation | Form A |
| Ethanol | x | ✓ | — | — | — | — | — | — | — | — | Maturation | Form A |
| Aceto- | x | x | x | x | x | x | x | x | x | x | Maturation | Form A |
| nitrile | | | | | | | | | | | | |
| Nitro- | x | x | x | x | x | x | x | x | x | x | Maturation | Form A |
| methane | | | | | | | | | | | | |
| Water | x | x | x | x | x | x | x | x | x | x | Maturation | Amorphous |
| 10% aq | ✓ | — | — | — | — | — | — | — | — | — | Maturation | Form A |
| acetone | | | | | | | | | | | | |
| 10% aq | x | ✓ | — | — | — | — | — | — | — | — | Maturation | Form A |
| MeOH | | | | | | | | | | | | |
| 10% aq | x | ✓ | — | — | — | — | — | — | — | — | Maturation | Form A |
| IPA | | | | | | | | | | | | |
| 10% aq | ✓ | ✓ | | | | | | | | | Slow evap. | Amorphous |
| THF | | | | | | | | | | | | |

✓: dissolved;
x: not dissolved.

### Example 3

### Slow Evaporation/Cooling Screens

The amorphous starting material (ca. 10 mg) was weighed into each vial and 50 volumes of solvent added (0.5 mL). If the solid fully dissolved, more material was added until slurries were obtained. The samples were then placed at 50° C. for one hour with additional solid added where necessary to ensure the samples remained slurries at this temperature. The slurries were then filtered to remove the excess solid from the saturated solutions. The collected solids were analyzed by XRPD (Table 3). The saturated solutions were then separated. One hundred microliters was pipetted into clean amber vials for slow cooling (Table 4) and 400 μL into clean amber vials for slow evaporation (Table 5). The samples for slow evaporation were stored at ambient temperature to allow the solvent to evaporate through a pin-hole. The slow cooling samples were stirred at 50° C. for one hour before cooling to 0° C. at 0.1° C./min. Any samples which remained as solutions at 0° C. were placed at −17° C. overnight, then anti-solvent (heptane) was added at ambient temperature if solutions persisted. All solids obtained were filtered (unless they had evaporated to dryness) under vacuum for analysis by XRPD. Those which had evaporated to dryness were removed by spatula.

TABLE 3
Analysis of Residual Solids from Preparation of Saturated Solutions

| Solvent | XRPD | Solvent | XRPD |
|---|---|---|---|
| Heptane | Amorphous | DCM | Not analyzed - |
| | | | glass |
| Dioxane | Amorphous | MEK | Form A |
| Toluene | Amorphous | Acetone | Form A |
| Transcutol | Not analyzed - glass | Methanol | Low Cryst. Form A |
| MTBE | Amorphous | Ethanol | Low Cryst. Form A |
| Tetraline | Amorphous | Acetonitrile | Form A |
| DIPE | Amorphous | Nitromethane | Form A |
| Anisole | Amorphous | Water | Amorphous |
| EtOAc | Form A | 10% aq acetone | Form A |
| IPAc | Form A | 10% aq MeOH | Low Cryst. Form A |
| IPA | Amorphous | 10% aq IPA | Low Cryst. Form A |
| THF | Form A | 10% aq THF | Amorphous |

TABLE 4
Analysis of Samples from Slow Cooling

| | Obs. On slow | Obs. At | Obs. on A/S | |
|---|---|---|---|---|
| Solvent | cooling to 0° C. | −17° C. | addn. | XRPD |
| Heptane | Solution | Solution | n/a | Glass |
| Dioxane | Solution | Frozen | Solid | Gum |
| Toluene | Slurry | | | Amorphous |
| Transcutol | Solution | Solution | No solid | |
| MTBE | Solution | Solution | No solid | |
| Tetraline | Slurry | | | Amorphous |
| DIPE | Solution | Solution | No solid | |
| Anisole | Slurry | | | Low cryst. |
| | | | | Form A |
| EtOAc | Slurry | | | Amorphous |
| IPAc | Slurry | | | Amorphous |
| IPA | Slurry | | | Amorphous |
| THF | Slurry | | | Form A |
| DCM | Solution | Solution | Solid | Amorphous |
| MEK | Solution | Solution | Solid | Amorphous |
| Acetone | Solution | Solution | Solid | Gum |
| Methanol | Slurry | | | Form A |
| Ethanol | Slurry | | | Amorphous |
| Acetonitrile | Solution | Solution | No solid | |
| Nitromethane | Solution | Solution | No solid | |
| Water | Solution | Frozen | No solid | |
| 10% aq | Slurry | | | Form A |
| acetone | | | | |
| 10% aq | Slurry | | | Low cryst. |
| MeOH | | | | Form A |
| 10% aq IPA | Slurry | | | Amorphous |
| 10% aq THF | Solution | Frozen | Solid | Gum |

TABLE 5
Analysis of Samples from Slow Evaporation

| Solvent | XRPD | Solvent | XRPD |
|---|---|---|---|
| Heptane | Glass | DCM | Amorphous solid |
| Dioxane | Glass | MEK | Glass |
| Toluene | Amorphous solid | Acetone | Glass |
| Transcutol | | Methanol | Amorphous solid |
| MTBE | Glass | Ethanol | Amorphous solid |
| Tetraline | | Acetonitrile | Glass |
| DIPE | Glass | Nitromethane | Glass |
| Anisole | Form A | Water | Too little an amount |
| EtOAc | Oil | 10% aq acetone | Amorphous solid |
| IPAc | Oil | 10% aq MeOH | Oil |
| IPA | Amorphous solid | 10% aq IPA | Oil |
| THF | Amorphous solid | 10% aq THF | Amorphous solid |

### Example 4

### Recrystallization Attempts in 15 Volumes Solvent on 100 mg Scale

One hundred milligrams of the amorphous form of the compound of Formula I was solubilized into 5 volumes of ethyl acetate or acetone under constant stirring at respectively 70° C. or 50° C. (experiment performed in duplicate). Solubilization was observed in less than 30 minutes. Systems were then allowed to equilibrate at initial temperature for 1 hour (crashing out reported in all cases). Some samples were then filtered at respectively 70° C. or 50° C. Other samples were cooled to 0° C. at 0.1° C./min. Suspensions were observed after 1 hour slurrying at 0° C. Ten volumes of solvent were added to each sample and the systems were allowed to equilibrate at 0° C. for 1 hour. One aliquot of each condition was filtered under reduced pressure, dried for 16 hours at 70° C. and 3 mbar and characterized by XRPD, ¹H-NMR and HPLC (Table 6). Analysis by GVS and storage at elevated temperature and humidity for eleven days had no effect on the crystals, which remained as Form A.

TABLE 6
Recrystallization Attempts in 15 Volumes Solvent on 100 mg Scale

| | | | | | | | Residual |
|---|---|---|---|---|---|---|---|
| | | | | | | | solvent |
| | | | | | | | (¹H |
| | | | | | | | NMR % |
| | | Time at | | | | | w/w) 16 hrs |
| | Initial | initial | Filtration | | | HPLC | at |
| Solvent | temp. | temp. | temp. | | XRPD | purity | 70° C., |
| (volume) | (° C.) | (hours) | (° C.) | Yield (%) | analysis | (%) | 3 mbar |
| Ethyl | 70 | 1 | 0 | 46 | Form A | 97.6 | 0.6 |
| acetate (15) | | | | | | | |
| Ethyl | 70 | 1 | 70 | 69 | Form A | 98.6 | 0.8 |
| acetate (15) | | | | | | | |
| Acetone (15) | 50 | 1 | 0 | 77 | Form A | 98.8 | 0.5 |
| Acetone (15) | 50 | 1 | 50 | 75 | Form A | 97.7 | 0.8 |

### Example 5

### Recrystallization in 15 Volumes Acetone on 10 g Scale

Ten grams of the compound of Formula I was added to 150 mL of acetone (15 volumes) in a jacketed flask at 50° C. and kept under stirring for 1 hour. A quick dissolution of the amorphous solid was reported in a few minutes followed by a precipitation in less than 1 hour. A sample from the corresponding suspension was filtered and characterized. The slurry was then cooled to 20° C. at 0.5° C./min and kept for 3 hours under stirring at 20° C. Filtration was performed at room temperature under reduced pressure (filter wash with 2 volumes of acetone) and the solid phase yielded was dried at 70° C. and 3 mbar for 16 hours prior to characterization by XRPD, HPLC and ¹H-NMR. Approximately 30-50 mg of initially dried material (16 hours at 70° C./3 mbar) were submitted to milling (Retsch ball mill MW300; 10 minutes at 10 Hz) prior to a second drying (65 hours at 70° C./3 mbar) and characterization (XRPD and if no decrease in crystallinity was detected ¹H-NMR and HPLC). The characterization results are summarized in Tables 7 and 8.

TABLE 7
Recrystallization in 15 Volumes Acetone on 10 g Scale

| | | | | | | | Residual |
|---|---|---|---|---|---|---|---|
| | | | | | | | solvent |
| | | | | | | | (¹H |
| | | | | | XRPD | HPLC | NMR % |
| | | Time at | | | analysis | purity (%) | w/w) |
| | Initial | initial | Filtration | | (before | (before | 16 hrs at |
| Solvent | temp. | temp. | temp. | Yield | and after | and after | 70° C., |
| (volume) | (° C.) | (hours) | (° C.) | (%) | milling) | milling) | 3 mbar |
| Acetone (15) | 50 | 1 | 20 | 91 | Form A | 98.6 | Before |
| | | | | | | | milling: |
| | | | | | | | 1.1 |
| | | | | | | | After |
| | | | | | | | milling: |
| | | | | | | | 0.4 |

TABLE 8
Analysis of Scaled-Up Form A

| | |
|---|---|
| XRPD | Form A |
| ¹H NMR | Consistent with structure + acetone |
| TGA | Wt loss of 1.7%, 90-225° C. |
| DSC | Melting endotherm at 247° C. |
| GVS | 1.16 wt % at 90% RH. Slight hysteresis on |
| | desorption above 60% RH, but not really |
| | hygroscopic |
| XRPD post GVS | Form A |
| TGA post GVS | 1.6% wt loss 100-240° C. |
| ¹H NMR post GVS | Acetone remained |
| Karl Fischer titration | 0.24% weight, 0.23% weight (in duplicate) |
| Stability 40° C./75% RH | Form A |
| (XRPD after 11 days) | |
| Stability 25° C./92% RH | Form A |
| (XRPD after 11 days) | |
| Thermodynamic Aqueous | Duplicate run: 0.03 mg/mL, 0.01 mg/mL |
| Solubility mgmL⁻¹ | (pH 6.42) |
| Microscopy | Tiny crystals <10 μm in length, |
| | agglomerates up to 50 μm |

### Example 6

The compound of Formula I (1.00 wt) and silica gel (0.4 wt) were charged in a vessel. Dichloromethane (13.3 wt) was added, followed by methanol (0.16 wt), and the mixture was stirred at 30-35° C. for 2 to 4 hrs. The mixture was filtered and the solids were washed with 2% methanol/dichloromethane. The washes and the filtrate were combined and silica gel (0.2 wt) was added. The mixture was stirred at 30-35° C. for 2 to 4 hrs. The mixture was filtered and the solids were washed with 2% methanol/dichloromethane.

The washes and the filtrate were combined and the volume was adjusted by distillation to ca. 3.5 vol. Ethanol (4.7 wt) was added and the volume of the mixture was again adjusted by distillation to ca. 3.5 volume. When the dichloromethane content reached no greater than 0.1% wt/wt dichloromethane v. ethanol, the mixture was cooled to 60-65° C. and toluene (3.5 wt) was added. The mixture was maintained at 60-65° C. for 4-5 hrs, cooled to 20-25° C. over 2-2.5 hrs, and maintained 20-25° C. over 2-3 hrs. The resulting slurry was filtered, the solids were washed with ethanol/toluene (3:4 v/v) and dried initially at about 23° C. under nitrogen purge and then 75° C. The yield was about 65-80% wt/wt.

### Example 7

The compound of Formula I (1.00 wt) and methanol (0.3 wt) were added to a vessel. Dichloromethane (13.3 wt) was added and the mixture was stirred at 30-35° C. until dissolution. After the solution was filtered through a 1 mm filter cartridge, it was heated to reflux and concentrated to ca. 5 vol. While maintaining reflux, acetone (35.6 wtwt) was added portionwise to maintain the total volume of the solution at about 10 vol. The mixture was cooled to 45-50° C. and additional acetone was added. When the dichloromethane content reached no greater than 12% wt/wt, the mixture was cooled to 15-20° C. over 2-3 hrs and maintained for additional 2-3 hrs. The slurry was filtered and the solids were washed with acetone and dried initially at about 23° C. and then 75° C. The yield was about 90-100% wt/wt.

### Example 8

### Salt Screen

### Experiments with 1 Equivalent of Acid

Ca. 50 mg of the compound of Formula I were weighed in a 2 mL amber vial, then 20 vol. of solvent added. Samples were then heated to 50° C. and equilibrated. 1 eq. of acid was added (from stock solution, 1M in THF) and the samples were allowed to equilibrate. The samples were then cooled to 5° C. at 0.1° C.min⁻¹. Solutions were divided in two aliquots of 500 μL. The first aliquot was allowed to evaporate at RT. 500 μL of anti-solvent (MTBE) was added to the second aliquot. Then samples were maturated at 4° C. for 2 days and then placed at −20° C. for 2 days. Suspension were filtered and analyzed by XRPD. If no evidence of crystalline salt was found, suspensions were placed in the maturation chamber RT/50° C. 8 h cycles. All solids were analyzed by XRPD or PLM to assess crystallinity. Thermodynamic aqueous solubility indicated solubility decreases as follows: HCl>H₂SO₄>tosylate=free base form. Results are summarized in Tables 9 and 10.

TABLE 9
Results of Experiments with 1 eq. of Acid

| | | | | Obs. .after | | | |
|---|---|---|---|---|---|---|---|
| Solvent | Acid | μL/mg | Obs. at 50° C. | addition. | Obs. at 5° C. | XRPD of solids | XRPD after maturation |
| MeOH | HBr | 56.5 | Sus | Sol | Sol | Gum | n/a |
| IPAc | HBr | 56.5 | Sus | Sus | Sus | Free base | Free base |
| Acetone | HBr | 56.5 | Sus | Sus | Sus | Free base | Free base |
| MeOH | HCl | 56.5 | Sus | Light sus | Sol | Gum | n/a |
| IPAc | HCl | 56.5 | Sus | Sus | Sus | Free base | Free base |
| Acetone | HCl | 56.5 | Sus | Sus | Sus | Free base | Free base |
| MeOH | H₂SO₄ | 56.5 | Sus | Sus | Gum | Amorphous | Amorphous |
| IPAc | H₂SO₄ | 56.5 | Sus | Sus | Sus | Free base | Free base |
| Acetone | H₂SO₄ | 56.5 | Sus | Sus | Sus | Amorphous | Amorphous |
| MeOH | p-Toluene | 56.5 | Sus | Sol | Sol | Gum | n/a |
| | sulphonic acid | | | | | | |
| IPAc | p-Toluene | 56.5 | Sus | Sus | Sus | Poorly crystalline | Free base |
| | sulphonic acid | | | | | | |
| Acetone | p-Toluene | 56.5 | Sus | Sus | Gum | Amorphous | Amorphous |
| | sulphonic acid | | | | | | |
| MeOH | Methane | 56.5 | Sus | Sol | Sol | Gum | n/a |
| | sulphonic acid | | | | | | |
| IPAc | Methane | 56.5 | Sus | Sus | Sus | Free base | Free base |
| | sulphonic acid | | | | | | |
| Acetone | Methane | 56.5 | Sus | Sus | Gum | Free base | Free base |
| | sulphonic acid | | | | | | |
| MeOH | Benzene | 56.5 | Sus | Sus | Sus | Amorphous | Amorphous |
| | sulphonic acid | | | | | | |
| IPAc | Benzene | 56.5 | Sus | Sus | Gum | Free base | Free base |
| | sulphonic acid | | | | | | |
| Acetone | Benzene | 56.5 | Sus | Sus | Sus | Poorly crystalline | Poorly crystalline |
| | sulphonic acid | | | | | | |
| MeOH | Oxalic acid | 56.5 | Sus | Light Sus | Light Sus | Gum | n/a |
| IPAc | Oxalic acid | 56.5 | Sus | Sus | Sus | Poorly crystalline | Poorly crystalline |
| Acetone | Oxalic acid | 56.5 | Sus | Sus | Sus | Free base | Free base |
| MeOH | L-Aspartic acid | 7.52 | Sus | Sus | Sus | Free base | Free base |
| IPAc | L-Aspartic acid | 7.52 | Sus | Sus | Sus | Free base + asp acid | Free base + asp acid |
| Acetone | L-Aspartic acid | 7.52 | Sus | Sus | Sus | Free base | Free base |
| MeOH | Maleic | 56.5 | Sus | Sol | Sol | Gum | n/a |
| | acid | | | | | | |
| IPAc | Maleic | 56.5 | Sus | Sus | Sus | Free base | Free base |
| | acid | | | | | | |
| Acetone | Maleic | 56.5 | Sus | Sus | Sus | Free base | Free base |
| | acid | | | | | | |
| MeOH | Ketoglutaric acid | 56.5 | Sus | Sus | Sus | Oil | n/a |
| IPAc | Ketoglutaric acid | 56.5 | Sus | Sus | Sus | Free base | Free base |
| Acetone | Ketoglutaric acid | 56.5 | Sus | Sus | Gum | Light Sus | n/a |
| MeOH | Malonic acid | 56.5 | Sus | Sus | Sus | Free base | Free base |
| IPAc | Malonic acid | 56.5 | Sus | Sus | Sus | Free base | Free base |
| Acetone | Malonic acid | 56.5 | Sus | Sus | Sus | Free base | Free base |
| MeOH | Thiocyanic acid | 56.5 | Sus | Sus | Sus | Free base | Free base |
| Acetone | Thiocyanic acid | 56.5 | Sus | Sus | Sus | Free base | Free base |

Legend: Sol, solution; Sus, suspension; n/a not applicable.

TABLE 10
Experiments with Anti-solvent for 1 eq. of Acid

| | | | | | | | Cooling | |
|---|---|---|---|---|---|---|---|---|
| | | | Anti- | | Obs. | Maturation | at | |
| Solvent | Vol./μL | Acid | solvent | Vol./μL | at RT | 5° C. | −20° C. | XRPD |
| MeOH | 500 | HBr | MTBE | 500 | Cloudy | Emu | Emu | Gum |
| MeOH | 500 | HCl | MTBE | 500 | Cloudy | Emu | Emu | Gum |
| MeOH | 500 | Tosylate | MTBE | 500 | Sol | Sol | Sol | Gum |
| MeOH | 500 | Mesylate | MTBE | 500 | Sol | Sol | Sol | Gum |
| MeOH | 500 | Oxalic acid | MTBE | 500 | Sus | Light Sus | Sus | Gum |
| MeOH | 500 | Maleic acid | MTBE | 500 | Sol | Sol | Sol | Gum |

Legend: Emu, emulsion; Sol, solution; Sus, suspension; n/a not applicable.

### Example 9

### Experiments with 2 Equivalents of Acid

Ca. 50 mg of the compound of Formula I was weighed in a 2 mL amber vial, then 10 vol. of solvent added. Samples were heated to 50° C. and equilibrated. 2 eq. of acid was added (from stock solution 1M in THF) and the samples were allowed to equilibrate. The samples were then cooled to 5° C. at 0.1° C.min⁻¹. Solutions were divided in two aliquots of 250 μL. The first aliquot was allowed to evaporate at RT. To the second up to 500 μL of anti-solvent (MTBE) were added. Then samples were then maturated at 4° C. for 2 days and then placed at −20° C. for 2 days. Suspensions were filtered and analyzed by XRPD. If no evidence of crystalline salt was found, suspensions were placed in the maturation chamber RT/50° C. 8 h cycles. All solids were analyzed by XRPD or PLM to assess crystallinity. Any hits were then further characterized by ¹H-NMR, IC and DSC. Evidence of amorphous salt formation for HBr, HCl, H₂SO₄, tosylate, mesylate, and besylate salts were found by ¹H-NMR. Results are summarized in Tables 11 to 13.

TABLE 11
Results for Experiments with 2 eq. of Acid

| | | | | | | XRPD after |
|---|---|---|---|---|---|---|
| Solvent | Counter-ion | Obs. at 50° C. | Obs. after add. | Obs. at 5° C. | XRPD of solids | maturation |
| MeOH | HBr | Sus | Sol | Sol | Gum | n/a |
| IPAc | HBr | Sus | Sus | Sus | Amorphous | Amorphous |
| Acetone | HBr | Sus | Sus | Gum | n/a | n/a |
| MeOH | HCl | Sus | Sol | Sol | Gum | Gum |
| IPAc | HCl | Sus | Sus | Sus | Amorphous | Amorphous |
| Acetone | HCl | Sus | Sus | Sus | Amorphous | Amorphous |
| MeOH | H₂SO₄ | Sus | Sol | Sol | Gum | n/a |
| IPAc | H₂SO₄ | Sus | Sus | Sus | Amorphous | Amorphous |
| Acetone | H₂SO₄ | Sus | Sus | Sus | Amorphous | Amorphous |
| MeOH | Tosylate | Sus | Sol | Sol | Gum | n/a |
| IPAc | Tosylate | Sus | Gum | Sus | Amorphous | Amorphous |
| Acetone | Tosylate | Sus | Light Sus | Gel | Gum | n/a |
| MeOH | Mesylate | Sus | Sol | Sol | Gum | n/a |
| IPAc | Mesylate | Sus | Sus | Sus | Amorphous | Amorphous |
| Acetone | Mesylate | Sus | Gum | Light Sus | Amorphous | Amorphous |
| MeOH | Besylate | Sus | Sol | Sol | Gum | n/a |
| IPAc | Besylate | Sus | Sus | Sus | Amorphous | Amorphous |
| Acetone | Besylate | Sus | Gum | Gel | Gum | n/a |
| MeOH | Oxalate | Sus | Sol | Sol | Gum | n/a |
| IPAc | Oxalate | Sus | Sus | Sus | Free base | Free base |
| Acetone | Oxalate | Sus | Sus | Sus | Amorphous | Amorphous |
| MeOH | Maleate | Sus | Sol | Sol | Gum | n/a |
| IPAc | Maleate | Sus | Sus | Sus | Free base | Free base |
| Acetone | Maleate | Sus | Light Sus | Gum | Gum | n/a |

Legend: Bold, evidence of salt formation; Sol, solution; Sus, suspension; n/a not applicable.

TABLE 12
Results of Anti-solvent Experiments for 2 eq. of Acid

| | | | | Obs. | | | | |
|---|---|---|---|---|---|---|---|---|
| | Counter- | Anti- | | at | Obs. after | Obs. at | | |
| Solvent | ion | solvent | μL | RT | add. | 5° C. | Obs. at −20° C. | Evaporation |
| MeOH | HBr | MTBE | 250 | Sus | White gum | Sol | Sol | Gum |
| | | | | | going | | | |
| | | | | | into | | | |
| | | | | | solution | | | |
| MeOH | HCl | MTBE | 250 | Sus | White gum | Sol | Sol | Gum |
| | | | | | going into | | | |
| | | | | | solution | | | |
| MeOH | H₂SO₄ | MTBE | 200 | Sus | Gum | Gum | Gum | Gum |
| MeOH | Tosylate | MTBE | 250 | Sus | White gum | Sol | Sol | Gum |
| | | | | | going into | | | |
| | | | | | solution | | | |
| MeOH | Mesylate | MTBE | 250 | Sus | White gum | Sol | Sol | Gum |
| | | | | | going into | | | |
| | | | | | solution | | | |
| MeOH | Besylate | MTBE | 250 | Sus | White gum | Sol | Sol | Gum |
| | | | | | going into | | | |
| | | | | | solution | | | |
| MeOH | Oxalate | MTBE | 150 | Sus | White gum | Sol | Sol | Gum |
| | | | | | going into | | | |
| | | | | | solution | | | |
| MeOH | Maleate | MTBE | 250 | Sus | White gum | Sol | Sol | Gum |
| | | | | | going into | | | |
| | | | | | solution | | | |

TABLE 13
Summary of Characterization of Selected Samples

| | Counter- | | | | | |
|---|---|---|---|---|---|---|
| Solvent | ion | XRPD of solids | ¹H NMR of solids | IC | Purity % | DSC |
| IPAc | HBr | Amorphous | Evidence of salt formation + | 1.6 | 98.8 | No relevant events observed |
| | | | IPAc | | | |
| IPAc | HCl | Amorphous | Evidence of salt formation + | 1.4 | 98.7 | No relevant events observed |
| | | | IPAc | | | |
| IPAc | H₂SO₄ | Amorphous | Evidence of salt formation + | 1.4 | 98.8 | No relevant events observed |
| | | | IPAc | | | |
| IPAc | Tosylate | Amorphous | Evidence of salt formation + | n/a | 98.0 | No relevant events observed |
| | | | IPAc + 2 eq. Tosylate | | | |
| IPAc | Mesylate | Amorphous | Evidence of salt formation + | n/a | 98.8 | No relevant events observed |
| | | | IPAc + 2 eq.Mesylate | | | |
| IPAc | Besylate | Amorphous | Evidence of salt formation + | n/a | 98.3 | No relevant events observed |
| | | | IPAc + 1 eq.Besylate | | | |
| Acetone | HCl | Amorphous | Evidence of salt formation + | 1.7 | 98.4 | No relevant events observed |
| | | | acetone | | | |
| Acetone | H₂SO₄ | Amorphous | Evidence of salt formation + | 1.4 | 98.5 | No relevant events observed |
| | | | acetone | | | |
| Acetone | Mesylate | Amorphous | Evidence of salt formation + | n/a | 98.7 | No relevant events observed |
| | | | acetone + 2eq. Mesylate | | | |

### Example 10

### Scale Up and Characterization

Ca. 0.5 g of the compound of Formula I was weighed in a 10 mL vial, 15 vol. of IPAc were added. The suspension was heated to 50° C., 2.5 eq of acid added (from a stock solution 1 M in THF) and then cooled to 5° C. at 0.1° C. min⁻¹. Suspension was filtered with vacuum and allowed to dry under these conditions for 4 h. Samples were analyzed by XRPD to check the conversion and then they were dried under vacuum at 30° C. for 20 h. The Tosylate salt showed evidence of free base and was recovered, then 15 vol. of IPAc added. The suspension was heated to 50° C. and additional 0.5 eq of acid added from a stock solution 1 M in THF. The suspension was then cooled to 5° C. at 0.1° C. min⁻¹. The solid was filtered with vacuum and allowed to dry under these conditions for 4 h. The solid was analyzed by XRPD to check the conversion and then it was dried under vacuum at 30° C. for 20 h. Full characterization was carried out for the three salts, HCl; H₂SO₄ and Tosylate, including additional stability of the salts after storage experiment (40° C./75% RH and 25° C./97% RH with/without ambient light exposure) and a solubility profile on the Free Base and HCl. Results are summarized in Table 14.

TABLE 14
Characterization of Scaled-Up Salts

| | | | |
|---|---|---|---|
| Form | HCl | H₂SO₄ | Tosylate |
| XRPD | Amorphous | Amorphous | Amorphous |
| ¹H NMR | Evidence salt | Evidence salt | Evidence salt |
| | formation + 0.11 eq. | formation + 0.28 eq. | formation + 0.14 eq. |
| | of IPAc | of IPAc | of IPAc |
| IC | 2.5 eq. | 1.5 eq. | 2 eq. |
| PLM | Irregular particles <50 μm | Irregular particles <50 μm | Irregular particles <50 μm |
| Purity | 98.9% area | 98.7% area | 97.8% area |
| TGA | 4.6% wt loss RT- | 5.2% wt loss RT- | 4.00% wt loss RT- |
| | 100° C. | 100° C. | 140° C. |
| | Decomposition at | Decomposition at | Decomposition at |
| | 160° C. | 140° C. | 200° C. |
| DSC | Endotherm at RT | Endotherm at RT | Endotherm at RT |
| | associated to solvent | associated to solvent | associated to solvent |
| | loss | loss | loss Endotherm at |
| | | | 137° C. associated to |
| | | | small presence of |
| | | | free base |
| mDSC | T_{g} at 20.6° C.(I) | T_{g} at 20.0° C.(I) | T_{g} at 22.9° C.(I) Event |
| | | | at 150° C. |
| VT-XRPD | n/a | n/a | No evident changes |
| | | | from RT to 160° C. |
| Karl Fischer titration | 1.75-2.94% wt of | 1.76-2.02% wt of | 0.89-1.35% wt of |
| | H₂O | H₂O | H₂O |
| Stability | Amorphous (Change | Amorphous (Change | Amorphous (Change |
| 40° C./75% RH | of color observed) | of color observed) | of color observed) |
| (XRPD after 7 days) | | | |
| Stability | Amorphous (Change | Amorphous (Change | Amorphous (Change |
| 25° C./92% RH | of color observed) | of color observed) | of color observed) |
| (XRPD after 7 days) | | | |
| Aqueous Solubility | 1.53 at pH 2.15 | 0.02 at pH 2.09 | 0.00 at pH 1.52 |
| mgmL⁻¹ | | | |
| GVS | 4.37% wt uptake | 6.12% wt uptake | 9.5% wt uptake |
| | between 40-90% RH | between 40-75% | between 0-90% RH |
| | 11.8% wt uptake | RH 14.5% wt | |
| | between 0-90% RH | uptake between 75-90% | |
| | | RH 25.5% wt | |
| | | uptake between 0-90% | |
| | | RH | |
| XRPD after GVS | Amorphous | Amorphous | Amorphous |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A crystalline form of a compound of methyl N-{(1R)-2-[(2S)-2-{5-[4-(6-{2-[(2S)-2-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate of Formula I

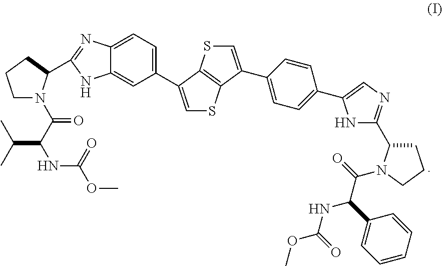

2. The crystalline form of claim 1, wherein the compound is a freebase.

3. The crystalline form of claim 1, wherein the crystalline form is Form A.

4-5. (canceled)

6. The crystalline form of claim 1, characterized by an X-ray powder diffraction pattern comprising peaks at approximately 2.7°, approximately 15.4°, and approximately 16.3°.

7-9. (canceled)

10. The crystalline form of claim 1, characterized by an X-ray powder diffraction pattern comprising two-theta peaks at approximately 2.7°, approximately 15.4°, approximately 16.3°, approximately 16.8°, approximately 19.7°, approximately 22.7°, and approximately 25.1°.

11. The crystalline form of claim 1, characterized by an X-ray powder diffraction pattern substantially as shown in FIG. 1.

12-14. (canceled)

15. The crystalline form of claim 1, having no less than about 90%, no less than about 91%, no less than about 92%, no less than about 93%, no less than about 94%, no less than about 95%, no less than about 96%, no less than about 97%, no less than about 98%, no less than about 98.2%, no less than about 98.5%, no less than about 98.7%, no less than 98.9%, no less than about 99%, or no less than about 99.5% by weight of the compound of Formula I.

16. (canceled)

17. The crystalline form of claim 1, having a residual solvent content no greater than about 5% by weight.

18-31. (canceled)

32. A pharmaceutically-acceptable salt of a compound of methyl N-{(1R)-2-[(2S)-2-{5-[4-(6-{2-[(2S)-1-{(2S)-2-[(methoxycarbonyl)amino]-3-methylbutanoyl}pyrrolidin-2-yl]-3H-benzimidazol-5-yl}thieno[3,2-b]thiophen-3-yl)phenyl]-1H-imidazol-2-yl}pyrrolidin-1-yl]-2-oxo-1-phenylethyl}carbamate of Formula I: 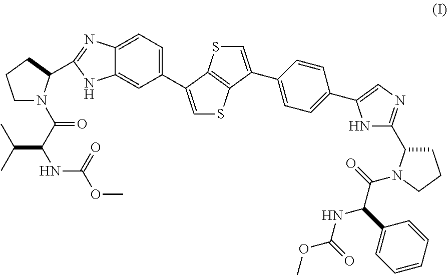
or a solvate thereof.

33. The pharmaceutically-acceptable salt of claim 32, wherein the pharmaceutically-acceptable salt is an acid addition salt.

34. The acid addition salt of claim 33, wherein the acid is selected from HI, HBr, HCl, HF, H₂SO₄, p-toluene sulfonic acid, methanesulfonic acid, benzenesulfonic acid, oxalic acid, L-aspartic acid, maleic acid, ketoglutaric acid, malonic acid, thiocyanic acid, L-tartaric acid, fumaric acid, citric acid, L-malic acid, D-gluconic acid, L-lactic acid, L-ascorbic acid, benzoic acid, nicotinic acid, glycolic acid, camphorsulfonic acid, sucrose, or nicotinamide.

35. (canceled)

36. The acid addition salt of claim 33, wherein the acid addition salt is crystalline or amorphous.

37-42. (canceled)

43. A pharmaceutical composition comprising the solid form of claim 1 and one or more pharmaceutically acceptable carriers.

44. The pharmaceutical composition of claim 43, further comprising a second antiviral agent.

45. The pharmaceutical composition of claim 44, wherein the second antiviral agent is selected from the group consisting of an interferon, ribavirin, an interleukin, an NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, and a ribozyme.

46-51. (canceled)

52. A method for treating HCV infection in a subject, which comprises administering to a subject the solid form of claim 1.

53-54. (canceled)

55. The method of claim 52, wherein the method comprises administering to the subject a second antiviral agent, in combination or alternation.

56. The method of claim 55, wherein the second antiviral agent is selected from the group consisting of an interferon, ribavirin, amantadine, an interleukin, a NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, and a ribozyme.

57-58. (canceled)

59. The method of claim 52, wherein the subject is a human.
